SEQUENCE LISTING (20 sequences)

SEQ ID NO: 1 - protein, 841 aa, Mus Musculus
MSCTFTALLRLGLTLSLWIPVLTGSLPKPILRVQPDSVVSRWTKVTFFCEETIGANEYRLYKDGKLYKTVTKNKQKPANKAEFSLSNVDLRNAGQYRCSYSTQYKSSGYSDPLELVVTGDYWTPSLLAQASPVVTSGGYVTLQCESWHNDHKFILTVEGPQKLSWTQDSQYNYSTRKYHALFSVGPVTPNQRWICRCYSYDRNRPYVWSPPSESVELLVSGNLQKPTIKAEPGPVIASKRAMTIWCQGNLDAEVYFLHNEGSQKTQSTQTLQQPGNKGKFFIPSMTRQHAGQYRCYCYGSAGWSQPSDTLELVVTGIYEHYKPRLSVLPSPVVTAGGNMTLHCASDFHYDKFILTKEDKKFGNSLDTEHISSSRQYRALFIIGPTTPTHTGTFRCYGYFKNAPQLWSVPSDLQQILISGLSKKPSLLTHQGHILDPGMTLTLQCYSDINYDRFALHKVGGADIMQHSSQQTDTGFSVANFTLGYVSSSTGGQYRCYGAHNLSSEWSASSEPLDILITGQLPLTPSLSVKPNHTVHSGETVSLLCWSMDSVDTFILSKEGSAQQPLRLKSKSHDQQSQAEFSMSAVTSHLSGTYRCYGAQNSSFYLLSSASAPVELTVSGPIETSTPPPTMSMPLGGLHMYLKALIGVSVAFILFLFILIFILLRRRHRGKFRKDVQKEKDLQLSSGAEEPITRKGELQKRPNPAAATQEESLYASVEDMQTEDGVELNSWTPPEEDPQGETYAQVKPSRLRKAGHVSPSVMSREQLNTEYEQAEEGQGANNQAAESGESQDVTYAQLCSRTLRQGAAASPLSQAGEAPEEPSVYATLAAARPEAVPKDVEQ

SEQ ID NO: 2 - protein, 598 aa, Homo Sapiens
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAYNLSSEWSAPSDPLDILITGQIHGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 3 - DNA, 22 bp, Artificial Sequence
  Primer
ggcataggtcacatcctgggac

SEQ ID NO: 4 - DNA, 21 bp, Artificial Sequence
  Primer
gtctcagaaaccattgaatcc

SEQ ID NO: 5 - DNA, 23 bp, Artificial Sequence
  Primer
gacagaaaactttgggtcatcag

SEQ ID NO: 6 - DNA, 22 bp, Artificial Sequence
  Primer
ccctcagcaacgatgaacaacc

SEQ ID NO: 7 - DNA, 22 bp, Artificial Sequence
  Primer
tggaccctggagtcagtgattc

SEQ ID NO: 8 - DNA, 24 bp, Artificial Sequence
  Primer
tgaaggctctcattggagtgtctg

SEQ ID NO: 9 - DNA, 22 bp, Artificial Sequence
  Primer
ggcataggtcacatcctgggac

SEQ ID NO: 10 - protein, 650 aa, Homo Sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 11 - protein, 652 aa, Homo Sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSAGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRQSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 12 - protein, 651 aa, Homo Sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSAGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 13 - protein, 651 aa, Homo Sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRQSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 14 - protein, 597 aa, Homo Sapiens
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 15 - protein, 632 aa, Homo Sapiens
MTPALTALLCLGLSLGPRTRVQAGPFPKPTLWAEPGSVISWGSPVTIWCQGSQEAQEYRLHKEGSPEPLDRNNPLEPKNKARFSIPSMTEHHAGRYRCHYYSSAGWSEPSDPLEMVMTGAYSKPTLSALPSPVVASGGNMTLRCGSQKGYHHFVLMKEGEHQLPRTLDSQQLHSRGFQALFPVGPVTPSHRWRFTCYYYYTNTPWVWSHPSDPLEILPSGVSRKPSLLTLQGPVLAPGQSLTLQCGSDVGYNRFVLYKEGERDFLQRPGQQPQAGLSQANFTLGPVSPSNGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSHPSEPLELVVSGHSGGSSLPPTGPPSTPGLGRYLEVLIGVSVAFVLLLFLLLFLLLRRQRHSKHRTSDQRKTDFQRPAGAAETEPKDRGLLRRSSPAADVQEENLYAAVKDTQSEDRVELDSQQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 16 - protein, 631 aa, Homo Sapiens
MTPALTALLCLGLSLGPRTRVQAGPFPKPTLWAEPGSVISWGSPVTIWCQGSQEAQEYRLHKEGSPEPLDRNNPLEPKNKARFSIPSMTEHHAGRYRCHYYSSAGWSEPSDPLEMVMTGAYSKPTLSALPSPVVASGGNMTLRCGSQKGYHHFVLMKEGEHQLPRTLDSQQLHSRGFQALFPVGPVTPSHRWRFTCYYYYTNTPWVWSHPSDPLEILPSGVSRKPSLLTLQGPVLAPGQSLTLQCGSDVGYNRFVLYKEGERDFLQRPGQQPQAGLSQANFTLGPVSPSNGGQYRCYGAHNLSSEWSAPSDPLNILMAGQIYDTVSLSAQPGPTVASGENVTLLCQSWWQFDTFLLTKEGAAHPPLRLRSMYGAHKYQAEFPMSPVTSAHAGTYRCYGSYSSNPHLLSHPSEPLELVVSGHSGGSSLPPTGPPSTPGLGRYLEVLIGVSVAFVLLLFLLLFLLLRRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 17 - protein, 591 aa, Homo Sapiens
MTLTLSVLICLGLSVGPRTCVQAGTLPKPTLWAEPASVIARGKPVTLWCQGPLETEEYRLDKEGLPWARKRQNPLEPGAKAKFHIPSTVYDSAGRYRCYYETPAGWSEPSDPLELVATGFYAEPTLLALPSPVVASGGNVTLQCDTLDGLLTFVLVEEEQKLPRTLYSQKLPKGPSQALFPVGPVTPSCRWRFRCYYYYRKNPQVWSNPSDLLEILVPGVSRKPSLLIPQGSVVARGGSLTLQCRSDVGYDIFVLYKEGEHDLVQGSGQQPQAGLSQANFTLGPVSRSHGGQYRCYGAHNLSPRWSAPSDPLDILIAGLIPDIPALSVQPGPKVASGENVTLLCQSWHQIDTFFLTKEGAAHPPLCLKSKYQSYRHQAEFSMSPVTSAQGGTYRCYSAIRSYPYLLSSPSYPQELVVSGPSGDPSLSPTGSTPTPAGPEDQPLTPTGLDPQSGLGRHLGVVTGVSVAFVLLLFLLLFLLLRHRHQSKHRTSAHFYRPAGAAGPEPKDQGLQKRASPVADIQEEILNAAVKDTQPKDGVEMDARAAASEAPQDVTYAQLHSLTLRREATEPPPSQEREPPAEPSIYAPLAIH

SEQ ID NO: 18 - protein, 590 aa, Homo Sapiens
MTLTLSVLICLGLSVGPRTCVQAGTLPKPTLWAEPASVIARGKPVTLWCQGPLETEEYRLDKEGLPWARKRQNPLEPGAKAKFHIPSTVYDSAGRYRCYYETPAGWSEPSDPLELVATGFYAEPTLLALPSPVVASGGNVTLQCDTLDGLLTFVLVEEEQKLPRTLYSQKLPKGPSQALFPVGPVTPSCRWRFRCYYYYRKNPQVWSNPSDLLEILVPGVSRKPSLLIPQGSVVARGGSLTLQCRSDVGYDIFVLYKEGEHDLVQGSGQQPQAGLSQANFTLGPVSRSHGGQYRCYGAHNLSPRWSAPSDPLDILIAGLIPDIPALSVQPGPKVASGENVTLLCQSWHQIDTFFLTKEGAAHPPLCLKSKYQSYRHQAEFSMSPVTSAQGGTYRCYSAIRSYPYLLSSPSYPQELVVSGPSGDPSLSPTGSTPTPGPEDQPLTPTGLDPQSGLGRHLGVVTGVSVAFVLLLFLLLFLLLRHRHQSKHRTSAHFYRPAGAAGPEPKDQGLQKRASPVADIQEEILNAAVKDTQPKDGVEMDARAAASEAPQDVTYAQLHSLTLRREATEPPPSQEREPPAEPSIYAPLAIH

SEQ ID NO: 19 - protein, 491 aa, Homo Sapiens
MTLTLSVLICLGLSVGPRTCVQAGTLPKPTLWAEPASVIARGKPVTLWCQGPLETEEYRLDKEGLPWARKRQNPLEPGAKAKFHIPSTVYDSAGRYRCYYETPAGWSEPSDPLELVATGVSRKPSLLIPQGSVVARGGSLTLQCRSDVGYDIFVLYKEGEHDLVQGSGQQPQAGLSQANFTLGPVSRSHGGQYRCYGAHNLSPRWSAPSDPLDILIAGLIPDIPALSVQPGPKVASGENVTLLCQSWHQIDTFFLTKEGAAHPPLCLKSKYQSYRHQAEFSMSPVTSAQGGTYRCYSAIRSYPYLLSSPSYPQELVVSGPSGDPSLSPTGSTPTPAGPEDQPLTPTGLDPQSGLGRHLGVVTGVSVAFVLLLFLLLFLLLRHRHQSKHRTSAHFYRPAGAAGPEPKDQGLQKRASPVADIQEEILNAAVKDTQPKDGVEMDARAAASEAPQDVTYAQLHSLTLRREATEPPPSQEREPPAEPSIYAPLAIH

SEQ ID NO: 20 - DNA, 24 bp, Artificial Sequence
  Primer
tgaaggctctcattggagtgtctg

(19) United States
(12) Patent Application Publication
(10) Pub. No.: US 2010/0047232 A1
(43) Pub. Date: Feb. 25, 2010

(54) MODULATORS OF NEURONAL REGENERATION

(76) Inventors: Jasvinder Atwal, San Carlos, CA (US); Marc Tessier-Lavigne, Woodside, CA (US)

Correspondence Address: Arnold & Porter LLP (24126);Attn: IP Docketing Dept., 555 Twelfth Street, N.W., Washington, DC, 20004-1206, US

(21) Appl. No.: 12/208,883

(22) Filed: Sep. 11, 2008

Related U.S. Application Data
(63) Continuation-in-part of application No. 11/983,775, filed on Nov. 9, 2007.
(60) Provisional application No. 60/865,772, filed on Nov. 14, 2006.
(60) Provisional application No. 60/890,416, filed on Feb. 16, 2007.

(51) Int. Cl. A61K 39/395; G01N 33/53; C07K 14/00; C07K 2/00; C07H 21/02; A61P 25/00

(52) U.S. Cl. 424/130.1; 435/7.1; 530/350; 530/300; 536/23.1

(57) ABSTRACT

The present invention provides methods and compositions related to CNS function and diseases.

### CROSS-REFERENCE TO RELATED APPLICATIONS

This application is a continuation-in-part of U.S. application Ser. No. 11/983,775 filed on Nov. 9, 2007, which claims priority under 35 U.S.C. 119(e) to Provisional Application No. 60/865,772 filed on Nov. 14, 2006 and Provisional Application No. 60/890,416 filed on Feb. 16, 2007, the entire disclosures of which are expressly incorporated by reference herein.

### FIELD OF THE INVENTION

The present invention relates generally to neural development and neurological disorders. The invention specifically concerns identification of novel modulators of the myelin-associated inhibitory system and various uses of the modulators so identified.

### BACKGROUND OF THE INVENTION

Myelin and Myelin-Associated Proteins

It is known that axons of the adult mammalian CNS neurons have very limited capacity to regenerate following injury, whereas axons in the peripheral nervous system (PNS) regenerate rapidly. CNS neuron's limited capacity to regenerate is in part an intrinsic property of CNS axons, but also due to an impermissible environment. The CNS myelin, while it is not the only source of inhibitory cues for neurite growth, contains numerous inhibitory molecules that actively block axonal growth and therefore constitutes a significant barrier to regeneration. Three of such myelin-associated proteins (MAPs) have been identified: Nogo (also known as NogoA) is a member of the Reticulon family of proteins having two transmembrane domains; myelin-associated glycoprotein (MAG) is a transmembrane protein of the Ig superfamily; and OMgp is a leucine rich repeat (LRR) protein with a glycosylphosphatidylinositol (GPI) anchor. Chen et al., Nature 403:434-39 (2000); GrandPre et al., Nature 417:439-444 (2000); Prinjha et al., Nature 403:383-384 (2000); McKerracher et al, Neuron 13:805-11 (1994); Wang et al, Nature 417:941-4 (20020: Kottis et al J. Neurochem 82:1566-9 (2002). A portion of NogoA, Nogo66, has been described as a 66-amino acid extracellular polypeptide that is found in all three isoforms of Nogo.

Despite their structural differences, all three inhibitory proteins (also Nogo66) have been shown to bind the same GPI-anchored receptor, called Nogo receptor (NgR; also known as Nogo Receptor-1 or NgR1), and it has been proposed that NgR might be required for mediating the inhibitory actions of Nogo, MAG and OMgp. Fournier et al., Nature 409:34′-346 (2001). Two NgR1 homologs (NgR2 and NgR3) have also been identified. US 2005/0048520 A1 (Strittmatter et al.), published Mar. 3, 2005. Given that NgR is a GPI-anchored cell surface protein, it is unlikely to be a direct signal transductor (Zheng et al., Proc. Natl. Acad. Sci. USA 102:1205-1210 (2005)). Others have suggested that the neurotrophin receptor p75^{NTK} acts as a co-receptor for NgR and provides the signal-transducing moiety in a receptor complex (Wang et al., Nature 420:74-78 (2002); Wong et al., Nat. Neurosci. 5:1302-1308 (2002)).

However, recent studies of the NgR/p75^{NTR} receptor complex have raised questions about NgR's role in the myelin-associated inhibitory system. Theng et al. have shown that genetic deletion of NgR does not reduce neurite inhibition in vitro or promote corticospinal tract (CST) regeneration in vivo. Zheng et al. (2005), supra. Consistent with these results, another study failed to detect any enhanced regeneration of the CST in NgR mutant mice. Kim et al. Neuron 44:439-451 (2004). These findings contradict the hypothesis that the NgR/p75^{NTR} receptor complex represents the key converging point for multiple inhibitory signals. The failure of CST regeneration in NgR mutant mice contrasts with the CST regeneration observed with wild-type animals treated with a peptide antagonist of the Nogo66/NgR interaction (GrandPre et al. Nature 417:5470551 (2002) and Li and Strittmatter, Nature 23:4219-4227 (2002)). Another study has shown that expression of a dominant-negative fragment of NgR lead to enhanced regeneration of optic nerve axons in combination with a conditional injury. Both these experiments failed to test directly the involvement of NgR, as both antagonistic peptides have the potential to interfere with other inhibitory ligands/receptors.

These inconsistencies with the experimental results are a strong indication that NgR, or the NgR/p75^{NTR} receptor complex, might play a limited role in the myelin associated inhibition of CNS regeneration, and other components, such as additional receptors or binding partners might participate in transmitting the inhibitory signal.

PirB and Human Orthologs

The major histocompatibility complex (MHC) class I was originally identified as a region encoding a family of molecules that are important for the immune system. Recent evidences have indicated that MHC class I molecules have additional functions in the development and adult CNS. Boulanger and Shatz, Nature Rev Neurosci. 5:521-531 (2004); US 2003/0170690 (Shatz and Syken), published Sep. 11, 2003. Many of the MHC class I members and their binding partners are found to be expressed in CNS neurons. Recent genetic and molecular studies have focused on the physiological functions of CNS MHC class I, and the initial results suggested that MHC class I molecules might be involved in activity-dependent synaptic plasticity, a process during which the strength of existing synaptic connections increases or decreases in response to neuronal activity, followed by long term structural alterations to circuits. Moreover, the MHC class 1 encoding region has also been genetically linked to a wide variety of disorders with neurological symptoms, and abnormal functions of MHC class I molecules are thought to contribute to the disruption of normal brain development and plasticity.

One of the known MHC class I receptors in the immune setting is PirB, a murine polypeptide that was first described by Kubagawa et al., Proc. Nat. Acad. Sci. USA 94:5261-6 (1997). Mouse PirB has several human orthologs, which are members of the leukocyte immunoglobulin-like receptor, subfamily B (LILRB), and are also referred to as “immunoglobulin-like transcripts” (ILTs). The human orthologs show significant homology to the murine sequence, from highest to lowest in the following order: LILRB3/ILT5, LILRB1/ILT2, LILRB5/ILT3, LILRB2/ILT4, and, just as PirB, are all inhibitory receptors. LILRB3/ILT5 (NP_{—}006855) and LILRB1/ILT2 (NP_{—}006660) were first described by Samaridis and Colonna, Eur. J. Immunol. 27(3):660-665 (1997). LILRB5/ILT3 (NP_{—}006831) has been identified by Borges et al., J. Immunol. 159(11):5192-5196 (1997). LILRB2/ILT4 (also known as M1R10), was identified by Colonna et al., J. Exp. Med. 186:1809-18 (1997). PirB and its human orthologs show a great degree of structural variability. The sequences of various alternatively spliced forms are available from EMBL/GenBank, including, for example, the following accession numbers for human ILT4 cDNA: ILT4-c11 AF009634; ILT4-c117 AF11566; ILT4-c126 AF11565. As noted above, the PirB/LILRB polypeptides are MHC Class I (MHCI) inhibitory receptors, and are known for their role in regulating immune cell activation (Kubagawa et al., supra; Hayami et al., J. Biol. Chem. 272:7320 (1997); Takai et al., Immunology 115:433 (2005); Takai et al., Immunol. Rev. 181:215 (2001); Nakamura et al. Nat. Immunol. 5:623 (2004); Liang et al., Eur. J. Immunol. 32:2418 (2002)).

A recent study by Syken et al. (Science 313:1795-800 (2006)) reported that PirB is expressed in subsets of neurons throughout the brain. In mutant mice lacking functional PirB, cortical ocular dominance (OD)) plasticity is significantly enhanced at all ages, suggesting PirB's function in restricting activity-dependent plasticity in visual cortex.

The present invention is based, at least in part, on the surprising finding that PirB/LILRB are binding partners for Nogo (Nogo66) and MAG, and that PirB/LILRB antagonists and reduced PirB/LILRB activity effectively disrupt the myelin-associated inhibitory pathway, thereby promoting neuronal regeneration.

### SUMMARY OF THE INVENTION

In one aspect, the invention concerns a method for identifying a PirB/LILRB antagonist comprising contacting a candidate agent with a receptor complex comprising PirB/LILRB and myelin or a myelin-associated protein, or a fragment thereof, and detecting the ability of the candidate agent to inhibit the interaction between PirB/LILRB and the myelin-associated protein, or fragment thereof, wherein the candidate agent is identified as an antagonist if the interaction is inhibited.

In one embodiment, the interaction detected is binding.

In another embodiment, the interaction detected is cellular signaling.

In a further embodiment, the cellular signaling results in the inhibition of axonal outgrowth or neuronal regeneration.

In a still further embodiment, the myelin-associated protein is selected from the group consisting of Nogo, MAG, OMgp, and fragments thereof.

In another embodiment, PirB/LILRB is a human LILRB protein, such as LILRB1, LILRB2, LILRB3, or LILRB5.

In certain specific embodiments, PirB/LILRB is selected from the group consisting of LILRB2, transcript variant 1 (SEQ ID NO: 2), LILRB2, transcript variant 2 (SEQ ID NO: 14), LILRB1, transcript variant 1 (SEQ ID NO: 10), LILRB1, transcript variant 2 (SEQ ID NO: 11), LILRB1, transcript variant 3 (SEQ ID NO: 12), LILRB1, transcript variant 4 (SEQ ID NO: 13), LILRB3, transcript variant 1 (SEQ ID NO: 15), LILRB3, transcript valiant 2 (SEQ ID NO: 16), LILRB5, transcript variant 1 (SEQ ID NO: 17). LILRB5, transcript variant 2 (SEQ ID NO: 18), and LILRB5, transcript variant 3 (SEQ ID NO: 19).

In an additional embodiment, receptor complex further comprises NgR.

In different embodiments, the candidate agent is selected from the group consisting of antibodies, polypeptides, peptides, nucleic acids, small organic molecules, polysaccharides and polynucleotides, and preferably is an antibody or a short-interfering RNA (siRNA). The antibody preferably specifically binds PirB/LILRB, such as LIRB2, and includes, without limitation, chimeric, humanized, human antibody and antibody fragments.

In a particular embodiment, the antibody fragment is elected from the group consisting of Fv, Fab, Fab′, and F(ab′)₂ fragments.

In a further embodiment, at least one of PirB/LILRB and the myelin or myelin-associated protein, or fragment thereof, is immobilized.

In a still further embodiment, the assay is a cell-based assay.

In a particular embodiment, the cell-based assay comprises culturing neuronal cells with the myelin or myelin-associated protein, of fragment thereof, in the presence and absence of a candidate agent and determining the change in neurite length, wherein the candidate agent is identified as an antagonist when the neurite length is longer in the presence of the candidate agent.

In the cell-based assay above, the neuronal cells may be primary neurons, or may, for example, be derived from cells or cell lines, including stem cells, e.g. embryonic stem (ES) cells. In other embodiments, the neurons may, for example, be selected from the group consisting of cerebellar granule neurons, dorsal root ganglion neurons, and cortical neurons.

In one embodiment, the methods described above further comprise the step of using the antagonist identified to enhance neurite outgrowth, and/or promoting neuronal growth, repair and/or regeneration.

In another embodiment, the methods described above further comprise the step of administering the antagonist identified to a subject with a disease or condition benefiting from the enhancement of neurite outgrowth, promotion of neuronal growth, repair or regeneration. Such disease or condition may, for example, be a neurological disorder, which may be characterized by a physically damaged nerve, or may be selected from the group consisting of peripheral nerve damage caused by physical injury, diabetes; physical damage to the central nervous system; brain damage associated with stroke, trigeminal neuralgia, glossopharyngeal neuralgia, Bell's Palsy, myasthenia gravis, muscular dystrophy, amyotrophic lateral sclerosis (ALS), progressive muscular atrophy, progressive bulbar inherited muscular atrophy, herniated, ruptured and prolapsed invertebrate disk syndromes, cervical spondylosis, plexus disorders, thoracic outlet destruction syndromes, peripheral neuropathies, prophyria, Gullain-Barre syndrome, Alzheimer's disease, Huntington's Disease, and Parkinson's disease.

In another aspect, the invention concerns an agent identified by any one of the methods herein.

In an embodiment, the agent is selected from the group consisting of antibodies, polypeptides, peptides, nucleic acids, small organic molecules, polysaccharides and polynucleotides, and preferably is an antibody or a short-interfering RNA (siRNA).

In a further aspect, the invention concerns a composition comprising an agent identified by the methods herein for stimulation of neuronal regeneration.

In a still further aspect, the invention concerns a kit comprising an agent identified by the methods herein and instructions for neuronal regeneration.

In yet another aspect, the invention concerns use of a complex of PirB/LILRB and myelin or a myelin-associated protein, or a fragment thereof, to identify a PirB/LILRB antagonist.

In a different aspect, the invention concerns use of a PirB/LILRB antagonist in the preparation of a medicament for the treatment of a disease or condition benefiting from the enhancement of neurite outgrowth, promotion of neuronal growth, repair or regeneration. In another aspect, the invention concerns use of a PirB/LILRB antagonist in the preparation of a medicament for the treatment of a neurological disorder, where the neurological disorder may be characterized by a physically damaged nerve, or may be selected, for example, from the group consisting of peripheral nerve damage caused by physical injury. diabetes: physical damage to the central nervous system; brain damage associated with stroke, trigeminal neuralgia, glossopharyngeal neuralgia, Bell's Palsy, myasthenia gravis, muscular dystrophy, amyotrophic lateral sclerosis (ALS), progressive muscular atrophy, progressive bulbar inherited muscular atrophy, herniated, ruptured and prolapsed invertebrate disk syndromes, cervical spondylosis, plexus disorders, thoracic outlet destruction syndromes, peripheral neuropathies, prophyria, Gullain-Barre syndrome, Alzheimer's disease, Huntington's Disease, and Parkinson's disease.

In a further aspect, the invention concerns a PirB/LILRB antagonist for use in the treatment of a disease or condition benefiting from the enhancement of neurite outgrowth, promotion of neuronal growth, repair or regeneration.

In a still further aspect, the invention concerns a PirB/LILRB antagonist for use in the treatment of a neurological disorder, where the neurological disorder is as discussed above.

### BRIEF DESCRIPTION OF THE DRAWINGS

FIG. 1 shows Alkaline Phosphatase activity on transfected COS cells following incubation with AP-Nogo66. AP-Nogo66 binds PirB and LILRB2.

FIG. 2A shows immunoreactivity on transfected COS cells following incubation with MAG-Fe. MAG binds PirB and LILRB2.

FIG. 2B shows immunoreactivity on transfected COS cells following incubation with AP-OMgp.

FIG. 3 shows RT-PCR results demonstrating the expression of SMAGP. PAN-PirA and PirB in various parts of the nervous system.

FIGS. 4A-4C confirm, by in situ hybridization, the expression of PirB in adult forebrain (A), adult cerebellum (B) and P10 Dorsal Root Ganglion (C).

FIG. 5 shows the mouse PirB amino acid sequence (SEQ ID NO: 1) and a human LILRB2, transcript variant 1 amino acid sequence (SEQ ID NO: 2).

FIG. 6 illustrates Nogo66 inhibition of axonal growth, and reverse of such inhibition by PirB ECD (both PirBFc and PirBHis). Cerebellar granule neurons were used for the assay.

FIG. 7 shows co-immunoprecipitation of PirB and NgR. NgR is robustly co-precipitated with PirB (left panel). The right panel shows total protein from whole cell lysates immunoblotted with anti-NgR. The multiple bands (arrows) represent NgR processed by glycosylation to varying extents.

FIG. 8A shows that Nogo66 inhibition of axonal growth is partially rescued by anti-PirB antibodies in cerebellar granule neurons.

FIG. 8B shows that an anti-PirB antibody binds PirB-transfected, but not untransfected, COS cells.

FIG. 9 shows the amino acid sequence of LILRB1, transcript variant 1 (SEQ ID NO: 10).

FIG. 10 shows the amino acid sequence of LILRB1, transcript variant 2 (SEQ ID NO: 11).

FIG. 11 shows the amino acid sequence of LILRB1, transcript variant 3 (SEQ ID so NO: 12).

FIG. 12 shows the amino acid sequence of LILRB1, transcript variant 4 (SEQ ID NO: 13).

FIG. 13 shows the amino acid sequence of LILRB2, transcript variant 2 (SEQ ID NO: 14).

FIG. 14 shows the amino acid sequence of LILRB3, transcript variant 1 (SEQ ID NO: 15).

FIG. 15 shows the amino acid sequence of LILRB3, transcript variant 2 (SEQ ID NO: 16).

FIG. 16 shows the amino acid sequence of LILRB5, transcript variant 1 (SEQ ID NO: 17).

FIG. 17 shows the amino acid sequence of LILRB5, transcript variant 2 (SEQ ID NO: 18).

FIG. 18 shows the amino acid sequence of LILRB5, transcript variant 3 (SEQ ID NO: 19).

### DETAILED DESCRIPTION OF THE INVENTION

### A. Definitions

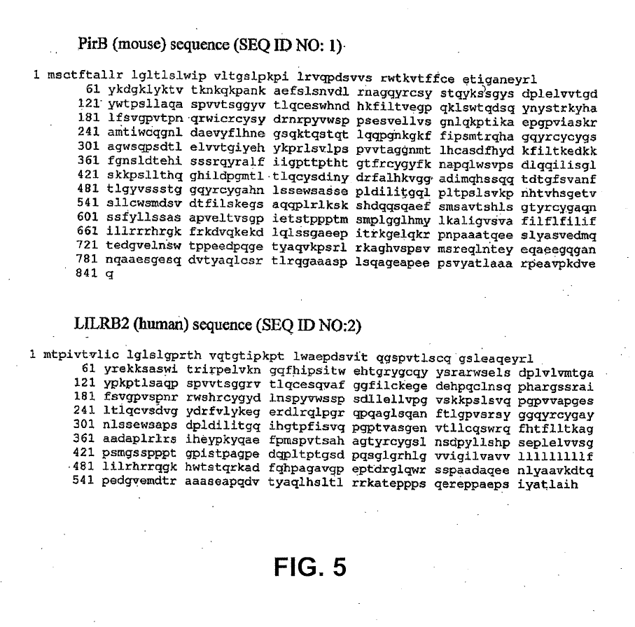

The terms “paired-immunoglobulin-like receptor B” and “PirB” are used herein interchangeably, and refer to a native-sequence, 841-amino acid mouse inhibitory protein of SEQ ID NO: 1 (FIG. 5) (NP_{—}035225), and its native-sequence homologues in rat and other non-human mammals, including all naturally occurring variants, such as alternatively spliced transcript variants and allelic variants and isoforms, as well as soluble forms thereof.

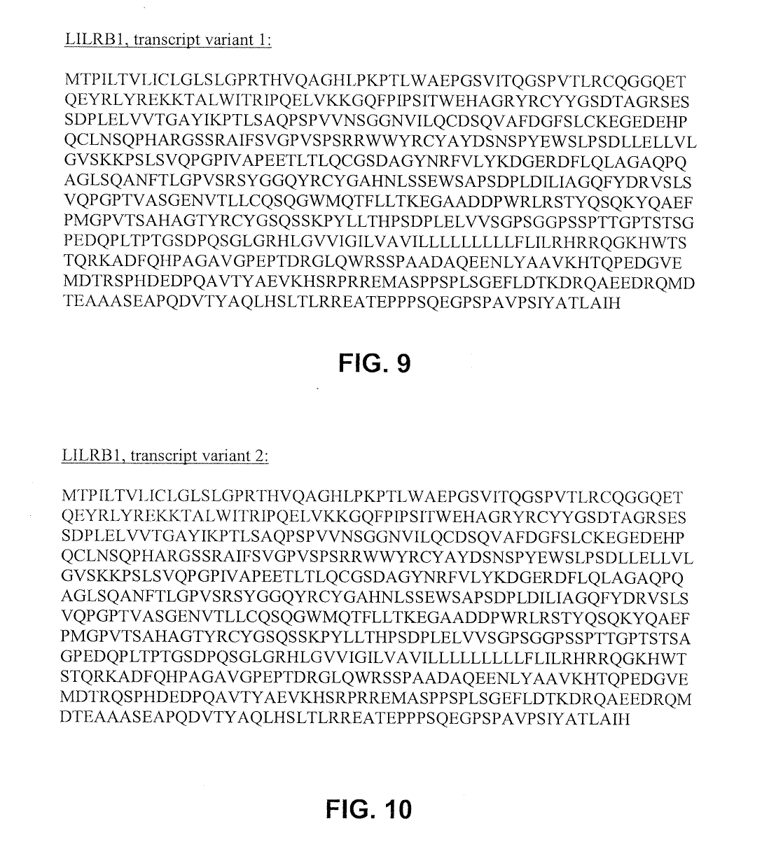

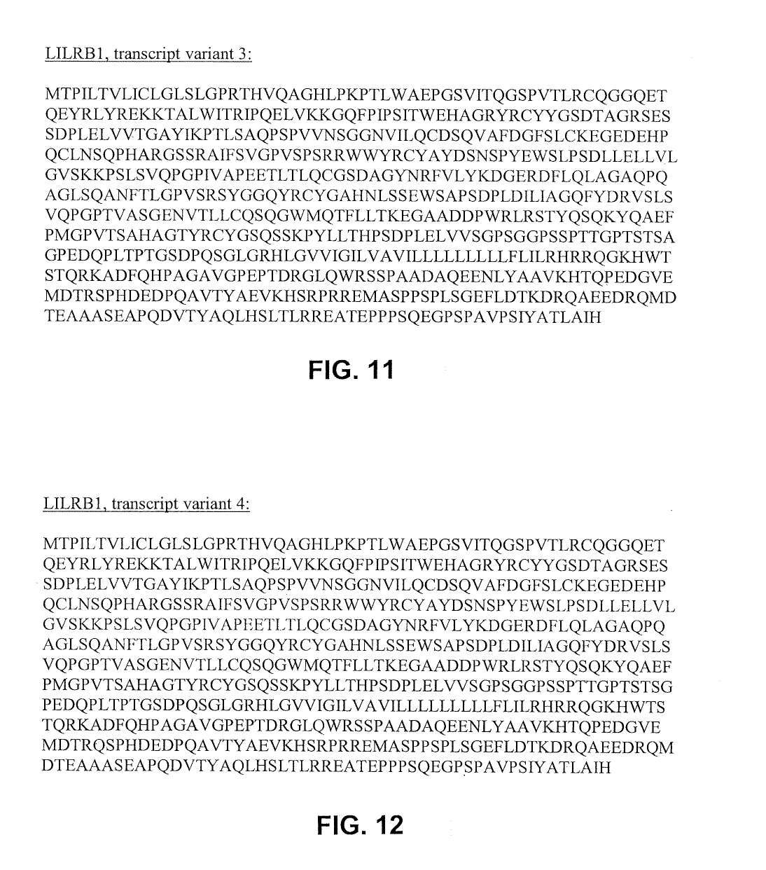

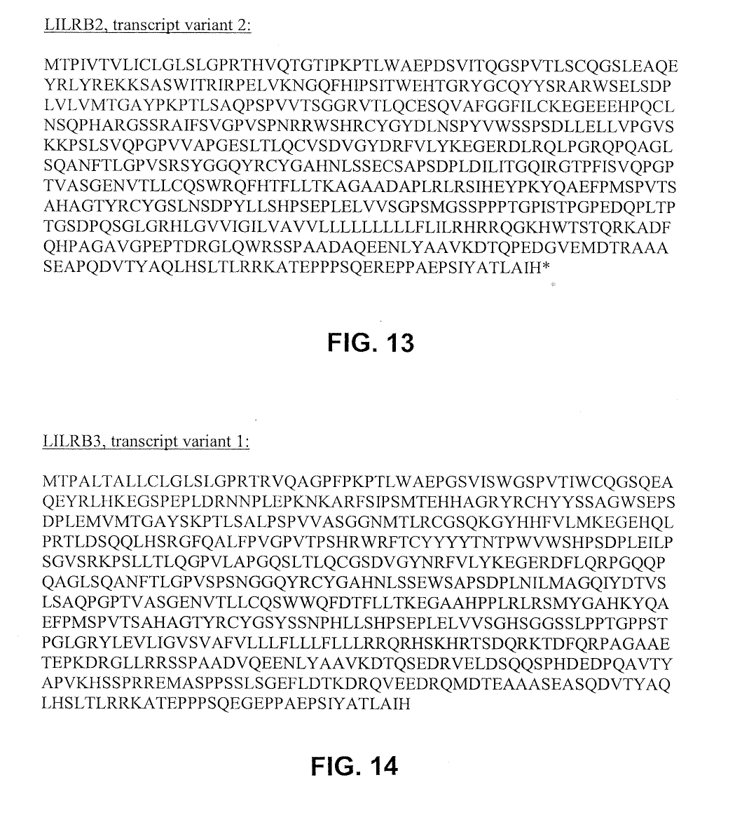

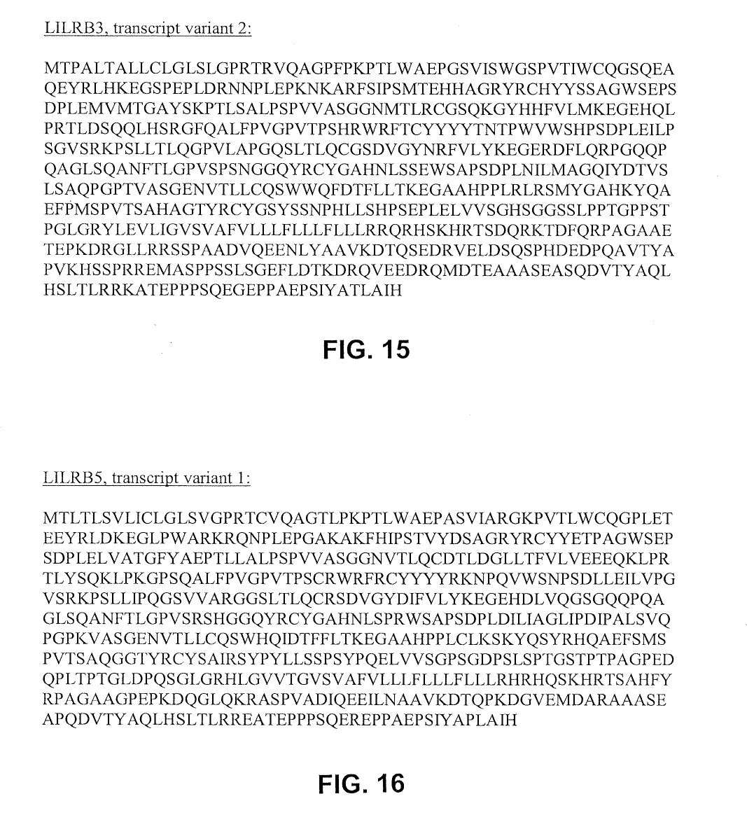

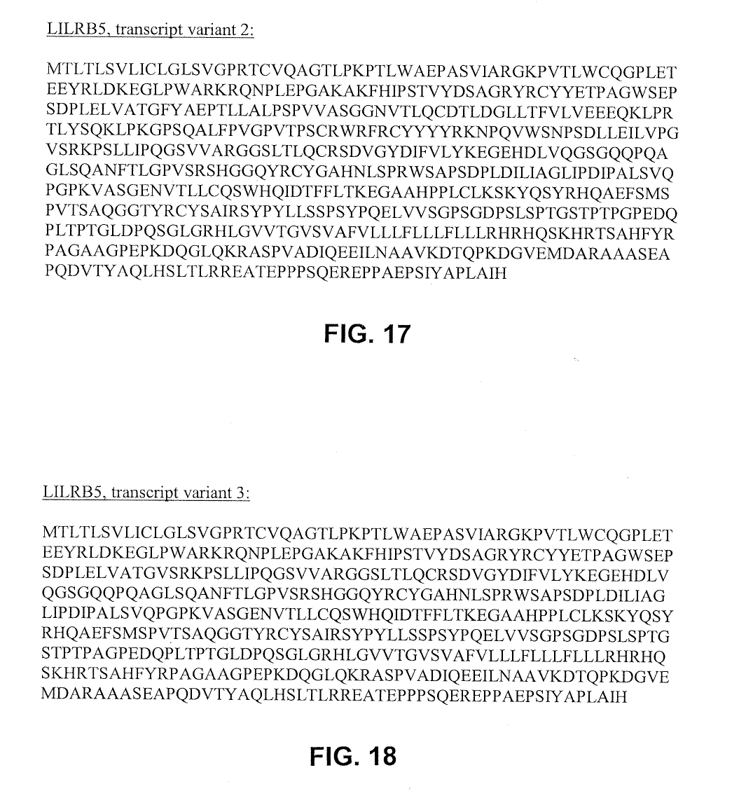

The terms “LILRB.” “ILT” and “MIR,” are used herein interchangeably, and refer to all members of the human “leukocyte immunoglobulin-like receptor, subfamily B”, including all naturally occurring variants, such as alternatively spliced transcript variants and allelic variants and isoforms, as well as soluble forms thereof. Individual members within this family are designated by numbers following the acronym, such as, for example, LILRB3/ILT5, LILRB1/ILT2, LILRB5/ILT3, and LILRB2/ILT4, where a reference to any individual member, unless otherwise noted, also includes reference to all naturally occurring variants, such as alternatively spliced transcript variants and allelic variants and isoforms, as well as soluble forms thereof. Thus, for example, “LILRB1” is used herein to specifically include transcript variants 1-4 (SEQ ID NOs: 10, 11, 12, and 13, shown in FIGS. 9-12), as well as all other naturally occurring variants, such as other alternatively spliced transcript variants, allelic variants and isoforms, and soluble forms thereof. The term “LILRB2” is used herein to specifically include LILRB2, transcript variant 1 (SEQ ID NO: 2, shown in FIG. 5) and transcript variant 2 (SEQ ID NO: 14, shown in FIG. 13), as well as all other naturally occurring variants, such as other alternatively spliced transcript variants, allelic variants and isoforms, and soluble forms thereof. The term “LILRB3” is used herein to specifically include LILRB3, transcript variant 1 (SEQ ID NO: 15, shown in FIG. 14) and transcript variant 2 (SEQ ID NO: 16, shown in FIG. 15), as well as all other naturally occurring variants, such as other alternatively spliced transcript variants, allelic variants and isoforms, and soluble forms thereof. The term “LILRB5” specifically includes transcript variants 1-3 (SEQ ID NOs: 17-19, shown in FIGS. 16-18), as well as all other naturally occurring variants, such as other alternatively spliced transcript variants, allelic variants and isoforms, and soluble forms thereof.

The term “PirB/LILRB” is used herein as a short-hand description to refer to any of the individual mouse PirB and human LILRB proteins and native sequence homologues in other non-human mammals, including all naturally occurring variants, such as alternatively spliced transcript and allelic variants and isoforms, as well as soluble forms thereof.

The term “myelin-associated protein” is used in the broadest sense and includes all proteins present in CNS myelin that inhibit neuronal regeneration, including Nogo, MAG and OMgp.

“Isolated,” when used to describe the various proteins disclosed herein, means protein that has been identified and separated and/or recovered from a component of its natural environment. Contaminant components of its natural environment are materials that would typically interfere with diagnostic or therapeutic uses for the protein, and may include enzymes, hormones, and other proteinaceous or non-proteinaceous solutes. In preferred embodiments, the protein will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain, or (3) to homogeneity by mass spectroscopic or peptide mapping techniques. Isolated protein includes protein in situ within recombinant cells, since at least one component of the natural environment of the protein in question will not be present. Ordinarily, however, isolated protein will be prepared by at least one purification step.

An “isolated” nucleic acid molecule is a nucleic acid molecule that is identified and separated from at least one contaminant nucleic acid molecule with which it is ordinarily associated in the natural source of the nucleic acid in question. An isolated nucleic acid molecule is other than in the form or setting in which it is found in nature. Isolated nucleic acid molecules therefore are distinguished from the nucleic acid molecules as they exist in natural cells. However, an isolated nucleic acid molecule includes nucleic acid molecules contained in cells that ordinarily express such nucleic acid where, for example, the nucleic acid molecule is in a chromosomal location different from that of natural cells.

As used herein, the term “PirB/LILRB antagonist” is used to refer to an agent capable of blocking, neutralizing, inhibiting, abrogating, reducing or interfering with PirB/LILRB activities. Particularly, the PirB/LILRB antagonist interferes with myelin associated inhibitory activities, thereby enhancing neurite outgrowth, and/or promoting neuronal growth, repair and/or regeneration. In a preferred embodiment, the PirB/LILRB antagonist inhibits the binding of PirB/LILRB to Nogo66 and/or MAG and/or OMgp by binding to PirB/LILRB. PirB/LILRB antagonists include, for example, antibodies to PirB/LILRB and antigen binding fragments thereof, truncated or soluble fragments of PirB/LILRB, Nogo 66, MAG or OMgp that are capable of sequestering the binding between PirB/LILRB and Nogo 66, or between PirB/LILRB and MAG, or between PirB/LILRB and OMgp and small molecule inhibitors of the PirB/LILRB related inhibitory pathway. PirB/LILRB antagonists also include short-interfering RNA (siRNA) molecules capable of inhibiting or reducing the expression of PirB/LILRB mRNA.

The term “antibody” herein is used in the broadest sense and specifically covers intact antibodies, monoclonal antibodies, polyclonal antibodies, multispecific antibodies (e.g. bispecific antibodies) formed from at least two intact antibodies, and antibody fragments, so long as they exhibit the desired biological activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al. Nature, 352:624-628 (1991) and Marks et al., J. Mol. Biol., 222:581-597 (1991), for example.

Antibodies specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include primatized antibodies comprising variable domain antigen-binding sequences derived from a non-human primate (e.g. Old World Monkey, Ape etc) and human constant region sequences.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragment(s).

An “intact” antibody is one which comprises an antigen-binding variable region as well as a light chain constant domain (C_{L}) and heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

“Humanized” forms of non-human (e.g., rodent) antibodies are chimeric antibodies that contain minimal sequence derived from non-human immunoglobulin. For the most pall, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains (Fab, Fab′, F(ab′)₂, Fabc, Fv), in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the regions of an antibody variable domain which are hypervariable in sequence and/or form structurally defined loops. The hypervariable region comprises amino acid residues from a “complementarity determining region” or “CDR” (i.e. residues 24-34, 50-56, and 89-97 in the light chain variable domain and 31-35, 50-65, and 95-102 in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32, 50-52, and 91-96 in the light chain variable domain and 26-32, 53-55, and 96-101 in the heavy chain variable domain; Chothia and Lesk J. Mol. Biol. 196:901-917 (1987)). In both cases, the variable domain residues are numbered according to Kabat et al., supra, as discussed in more detail below. “Framework” or “FR” residues are those variable domain residues other than the residues in the hypervariable regions as herein defined.

A “parent antibody” or “wild-type” antibody is an antibody comprising an amino acid sequence which lacks one or more amino acid sequence alterations compared to an antibody variant as herein disclosed. Thus, the parent antibody generally has at least one hypervariable region which differs in amino acid sequence from the amino acid sequence of the corresponding hypervariable region of an antibody variant as herein disclosed. The parent polypeptide may comprise a native sequence (i.e. a naturally occurring) antibody (including a naturally occurring allelic variant), or an antibody with pre-existing amino acid sequence modifications (such as insertions, deletions and/or other alterations) of a naturally occurring sequence. Throughout the disclosure, “wild type,” “WT,” “wt,” and “parent” or “parental” antibody are used interchangeably.

As used herein, “antibody variant” or “variant antibody” refers to an antibody which has an amino acid sequence which differs from the amino acid sequence of a parent antibody. Preferably, the antibody variant comprises a heavy chain variable domain or a light chain variable domain having an amino acid sequence which is not found in nature. Such variants necessarily have less than 100% sequence identity or similarity with the parent antibody. In a preferred embodiment, the antibody variant will have an amino acid sequence from about 75% to less than 100% amino acid sequence identity or similarity with the amino acid sequence of either the heavy or light chain variable domain of the parent antibody, more preferably from about 80% to less than 100%, more preferably from about 85% to less than 100%, more preferably from about 90% to less than 100%, and most preferably from about 95% to less than 100%. The antibody variant is generally one which comprises one or more amino acid alterations in or adjacent to one or more hypervariable regions thereof.

An “amino acid alteration” refers to a change in the amino acid sequence of a predetermined amino acid sequence. Exemplary alterations include insertions, substitutions and deletions. An “amino acid substitution” refers to the replacement of an existing amino acid residue in a predetermined amino acid sequence; with another different amino acid residue.

A “replacement” amino acid residue refers to an amino acid residue that replaces or substitutes another amino acid residue in an amino acid sequence. The replacement residue may be a naturally occurring or non-naturally occurring amino acid residue.

An “amino acid insertion” refers to the introduction of one or more amino acid residues into a predetermined amino acid sequence. The amino acid insertion may comprise a “peptide insertion” in which case a peptide comprising two or more amino acid residues joined by peptide bond(s) is introduced into the predetermined amino acid sequence. Where the amino acid insertion involves insertion of a peptide, the inserted peptide may be generated by random mutagenesis such that it has an amino acid sequence which does not exist in nature. An amino acid alteration “adjacent a hypervariable region” refers to the introduction or substitution of one or more amino acid residues at the N-terminal and/or C-terminal end of a hypervariable region, such that at least one of the inserted or replacement amino acid residue(s) form a peptide bond with the N-terminal or C-terminal amino acid residue of the hypervariable region in question.

A “naturally occurring amino acid residue” is one encoded by the genetic code, generally selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val).

A “non-naturally occurring amino acid residue” herein is an amino acid residue other than those naturally occurring amino acid residues listed above, which is able to covalently bind adjacent amino acid residues(s) in a polypeptide chain. Examples of non-naturally occurring amino acid residues include norleucine, ornithine, norvaline, homoserine and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA.

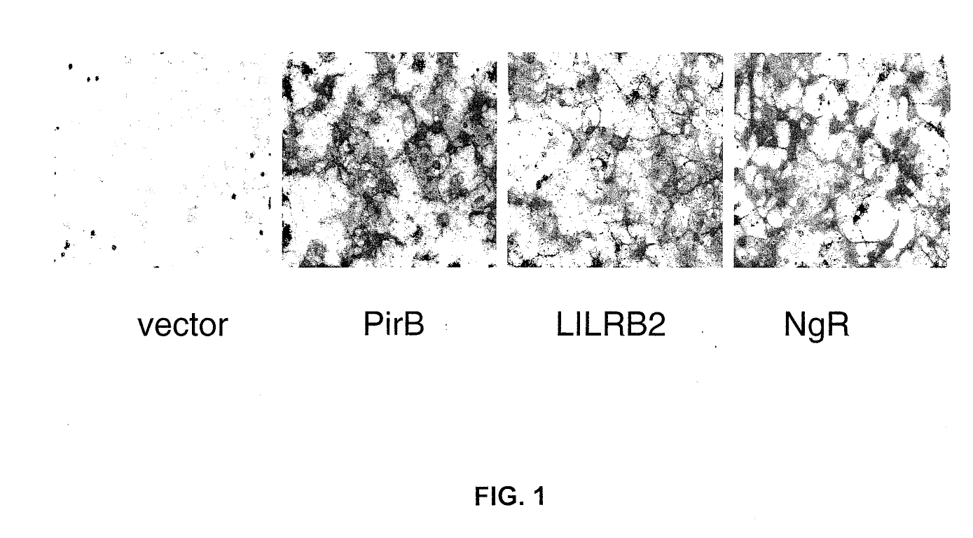

Throughout this disclosure, reference is made to the numbering system from Kabat, E. A., et al., Sequences of Proteins of Immunological Interest (National Institutes of Health, Bethesda, Md. (1987) and (1991). In these compendiums, Kabat lists many amino acid sequences for antibodies for each subclass, and lists the most commonly occurring amino acid for each residue position in that subclass. Kabat uses a method for assigning a residue number to each amino acid in a listed sequence, and this method for assigning residue numbers has become standard in the field. The Kabat numbering scheme is followed in this description. For purposes of this invention, to assign residue numbers to a candidate antibody amino acid sequence which is not included in the Kabat compendium, one follows the following steps. Generally, the candidate sequence is aligned with any immunoglobulin sequence or any consensus sequence in Kabat. Alignment may be done by hand, or by computer using commonly accepted computer programs; an example of such a program is the Align 2 program. Alignment may be facilitated by using some amino acid residues which are common to most Fab sequences. For example, the light and heavy chains each typically have two cysteines which have the same residue numbers; in V_{L} domain the two cysteines are typically at residue numbers 23 and 88, and in the V_{H} domain the two cysteine residues are typically numbered 22 and 92. Framework residues generally, but not always, have approximately the same number of residues, however the CDRs will vary in size. For example, in the case of a CDR from a candidate sequence which is longer than the CDR in the sequence in Kabat to which it is aligned, typically suffixes are added to the residue number to indicate the insertion of additional residues (see, e.g. residues 100abc in FIG. 1B). For candidate sequences which, for example, align with a Kabat sequence for residues 34 and 36 but have no residue between them to align with residue 35, the number 35 is simply not assigned to a residue.

As used herein, an antibody with a “high-affinity” is an antibody having a K_{D}, or dissociation constant, in the nanomolar (nM) range or better. A K_{D} in the “nanomolar range or better” may be denoted by X nM, where X is a number less than about 10.

The term “filamentous phage” refers to a viral particle capable of displaying a heterogenous polypeptide on its surface, and includes, without limitation, fl, fd, Pfl, and Ml3. The filamentous phage may contain a selectable marker such as tetracycline (e.g., “fd-tet”). Various filamentous phage display systems are well known to those of skill in the art (see, e.g., Zacher et al. Gene 9: 127-140 (1980), Smith et al. Science 228:1315-1317 (1985); and Parmley and Smith Gene 73:305-318 (1988)).

The term “panning” is used to refer to the multiple rounds of screening process in identification and isolation of phages carrying compounds, such as antibodies, with high affinity and specificity to a target.

The term “short-interfering RNA (siRNA)” refers to small double-stranded RNAs that interfere with gene expression. siRNAs are an intermediate of RNA interference, the process double-stranded RNA silences homologous genes. siRNAs typically are comprised of two single-stranded RNAs of about 15-25 nucleotides in length that form a duplex, which may include single-stranded overhang(s). Processing of the double-stranded RNA by an enzymatic complex, for example by polymerases, results in the cleavage of the double-stranded RNA to produce siRNAs. The antisense strand of the siRNA is used by an RNA interference (RNAi) silencing complex to guide mRNA cleavage, thereby promoting mRNA degradation. To silence a specific gene using siRNAs, for example in a mammalian cell, the base pairing region is selected to avoid chance complementarity to an unrelated mRNA. RNAi silencing complexes have been identified in the art, such as, for example, by Fire et al: Nature 391:806-811 (1998) and McManus et al., Nat. Rev. Genet. 3(10):737-47 (2002).

The term “interfering RNA (RNAi)” is used herein to refer to a double-stranded RNA that results in catalytic degradation of specific mRNAs, and thus can be used to inhibit/lower expression of a particular gene.

The term “polymorphism” is used herein to refer to more than one forms of a gene or a portion (e.g., allelic variant) thereof. A portion of a gene of which there are at least two different forms is referred to as a “polymorphic region” of the gene. A specific genetic sequence at a polymorphic region of a gene is an “allele.” A polymorphic region can be a single nucleotide, which differs in different alleles, or can be several nucleotides long.

As used herein, the term “disorder” in general refers to any condition that would benefit from treatment with the compounds of the present invention, including any disease or disorder that can be treated by effective amounts of antagonists of PirB/LILRB. Non-limiting examples of disorders to be treated herein include, without limitation, diseases and conditions benefiting from the enhancement of neurite outgrowth, promotion of neuronal growth, repair or regeneration, including neurological disorders, such as physically damaged nerves and neurodegenerative diseases. Such disorders specifically include peripheral nerve damage caused by physical injury or disease states such as diabetes: physical damage to the central nervous system (spinal cord and brain); brain damage associated with stroke; and neurological disorders relating to neurodegeneration, such as, for example, trigeminal neuralgia, glossopharyngeal neuralgia, Bell's Palsy, myasthenia gravis, muscular dystrophy, amyotrophic lateral sclerosis (ALS), progressive muscular atrophy, progressive bulbar inherited muscular atrophy, herniated, ruptured or prolapsed invertebrate disk syndromes, cervical spondylosis, plexus disorders, thoracic outlet destruction syndromes, peripheral neuropathies such as those caused by lead, dapsone, ticks, prophyria, Gullain-Barre syndrome, Alzheimer's disease, Huntington's Disease, or Parkinson's disease.

The terms “treating”, “treatment” and “therapy” as used herein refer to curative therapy, prophylactic therapy, and preventative therapy. Consecutive treatment or administration refers to treatment on at least a daily basis without interruption in treatment by one or more days. Intermittent treatment or administration, or treatment or administration in an intermittent fashion, refers to treatment that is not consecutive, but rather cyclic in nature.

The term “preventing neurodegeneration,” as used herein includes (1) the ability to inhibit or prevent neurodegeneration in patients newly diagnosed as having a neurodegenerative disease or at risk of developing a new neurodegenerative disease and (2) the ability to inhibit or prevent further neurodegeneration in patients who are already suffering from, or have symptoms of. a neurodegenerative disease.

The term “mammal” as used herein refers to any mammal classified as a mammal, including humans, higher non-human primates, rodents, domestic and farm animals, such as cows, horses, dogs and cats. In a preferred embodiment of the invention, the mammal is a human.

Administration “in combination with” one or more further therapeutic agents includes simultaneous (concurrent) and consecutive administration in any order.

An “effective amount” is an amount sufficient to effect beneficial or desired therapeutic (including preventative) results. An effective amount can be administered in one or more administrations.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. The term “progeny” refers to any and all offspring of every generation subsequent to an originally transformed cell or cell line. Mutant progeny that have the same function or biological activity as screened for in the originally transformed cell are included. Where distinct designations are intended, it will be clear from the context.

“Percent (%) amino acid sequence identity” with respect to the sequences identified herein is defined as the percentage of amino acid residues in a candidate sequence that are identical with the amino acid residues in a reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art can determine appropriate parameters for measuring alignment, including assigning algorithms needed to achieve maximal alignment over the full-length sequences being compared. For purposes herein, percent amino acid identity values can be obtained using the sequence comparison computer program, ALIGN-2, which was authored by Genentech, Inc. and the source code of which has been filed with user documentation in the US Copyright Office, Washington, D.C., 20559, registered under the US Copyright Registration No. TXU510087. The ALIGN-2 program is publicly available through Genentech, Inc., South San Francisco, Calif. All sequence comparison parameters are set by the ALIGN-2 program and do not vary.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired identity between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“High stringency conditions”, as defined herein, are identified by those that: (1) employ low ionic strength and high temperature for washing; 0.015 M sodium chloride/0.0015 M sodium citrate/0.1% sodium dodecyl sulfate at 50° C.; (2) employ during hybridization a denaturing agent; 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

A “small molecule” is defined herein to have a molecular weight below about 1000 Daltons, preferably below about 500 Daltons.

### B. Screening Assays to Identify Stimulators of Neuronal Regeneration

The primary assays of the present invention are at least in part based on the recognition that PirB/LILRB is a receptor of the myelin proteins Nogo (Nogo66) and MAG, and that PirB/LILRB antagonists, which interfere with the association of PirB/LILRB with Nogo and/or MAG, are capable of enhancing neurite outgrowth, and/or promoting neuronal growth, repair and/or regeneration. In brief, such agents will be referred to herein as stimulators of neuronal regeneration.

Screening assays for antagonist drug candidates may be designed to identify compounds that bind or complex with PirB/LILRB, or otherwise interfere with the interaction of PirB/LILRB with Nogo, MAG or other members of the myelin-associated inhibitory system. The screening assays provided herein include assays amenable to high-throughput screening of chemical libraries, making them suitable for identifying small molecule drug candidates. Generally, binding assays and activity assays are provided.

The assays can be performed in a variety of formats, including, without limitation, protein-protein binding assays, biochemical screening assays, immunoassays, and cell-based assays, which are well characterized in the art.

All assays for antagonists are common in that they call for contacting the drug candidate with a PirB/LILRB polypeptide under conditions and for a time sufficient to allow these two components to interact.

In binding assays, the interaction is binding, and the complex formed can be isolated or detected in the reaction mixture. In a particular embodiment, either the PirB/LILRB polypeptide or the drug candidate is immobilized on a solid phase, e.g., on a microtiter plate, by covalent or non-covalent attachments. Non-covalent attachment generally is accomplished by coating the solid surface with a solution of the PirB/LILRB polypeptide and drying. Alternatively, an immobilized antibody, e.g., a monoclonal antibody, specific for the PirB/LILRB polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound is a polypeptide which interacts with but does not bind to PirB/LILRB, the interaction of PirB/LILRB with the respective polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of PirB/LILRB and other intra- or extracellular components, in particular Nogo or MAG can be tested as follows. Usually a reaction mixture is prepared containing PirB/LILRB and the intra- or extracellular component under conditions and for a time allowing for the interaction of the two products. To test the ability of a candidate compound to inhibit the interaction of PirB/LILRB and Nogo or MAG, the reaction is run in the absence and in the presence of the test compound. In addition, a placebo may be added to a third reaction mixture, to serve as positive control.

It is emphasized that the screening assays specifically discussed herein are for illustration only. A variety of other assays, which can be selected depending on the type of the antagonist candidates screened (e.g. polypeptides, peptides, non-peptide small organic molecules, nucleic acid, etc.) are well know to those skilled in the art and are equally suitable for the purposes of the present invention.

The assays herein may be used to screed libraries of compounds, including, without limitation, chemical libraries, natural product libraries (e.g. collections of microorganisms, animals, plants, etc.), and combinatorial libraries comprised of random peptides, oligonucleotides or small organic molecules. In a particular embodiment, the assays herein are used to screen antibody libraries, including, without limitation, naïve human, recombinant, synthetic and semi-synthetic antibody libraries. The antibody library can, for example, be a phage display library, including monovalent libraries, displaying on average one single-chain antibody or antibody fragment per phage particle, and multi-valent libraries, displaying, on average, two or more antibodies or antibody fragments per viral particle. However, the antibody libraries to be screened in accordance with the present invention are not limited to phage display libraries. Other display technique include, for example, ribosome or mRNA display (Mattheakis et al., Proc. Natl. Acad. Sci. USA 91:9022-9026 (1994); Hanes and Pluckthun, Proc. Natl. Acad. Sci. USA 94:4937-4942 (1997)), microbial cell display, such as bacterial display (Georgiou et al., Nature Biotech. 15:29-34 (1997)), or yeast cell display (Kieke at al., Protein Eng. 10:1303-1310 (1997)), display on mammalian cells, spore display, viral display, such as retroviral display (Urban et al., Nucleic Acids Res. 33:e35 (2005), display based on protein-DNA linkage (Odegrip et al., Proc. Acad. Natl. Sci. USA 101:2806-2810 (2004); Reiersen et al., Nucleic Acids Res. 33:e10 (2005)), and microbead display (Sepp et al., FEBS Lett. 532:455-458 (2002)).

The results obtained in the primary binding/interaction assays herein can be confirmed in in vitro and/or in vivo assays of neuronal regeneration. Alternatively, in vitro and/or in vivo assays of neuronal regeneration may be used as primary assays to identify the PirB/LILRB antagonists herein.

In vitro assays of neurite outgrowth are well known in the art and are described, for example, Jin and Strittmatter, J Neurosci 17:6256-6263 (1997); Fournier et al., Methods Enzymol. 325:473-482 (2000); Zheng et al., Neuron 38:213-224 (2003); Wang et al., Nature 417:941-944 (2002), and Neumann et al., Neuron 34:885-893 (2002)). Kits for measuring and quantifying neurite outgrowth are commercially available. Thus, for example, CHEMICON's Neurite Outgrowth Assay Kit (Catalog number NS200), uses microporous filter technology for the quantitative testing of compounds that influence neurite formation and repulsion. With this system, it is possible to screen biological and pharmacological agents simultaneously, directly evaluate adhesion and guidance receptor functions responsible for neurite extension and repulsion, as well as the analysis of gene function in transfected cells. The microporous filter allows for biochemical separation and purification of neurites and cell bodies for detailed molecular analysis of protein expression, signal transduction processes and identification of drug targets that regulate neurite outgrowth or retraction processes.

In a typical protocol, primary neurons isolated from rodent neural tissue (including cerebellar granule neurons, dorsal root ganglion neurons, and cortical neurons) are cultured on 96-well tissue culture dishes coated with immobilized whole myelin or myelin associated proteins (e.g. Nogo66, MAG and/or OMgp). Following a defined time in culture, typically 24-48 hours, the neurons are fixed with 4% paraformaldehyde and stained with a neuronal marker (anti-class III b-Tubulin, Covance). Image acquisition and analysis are then performed using the ImageXpress automated imaging system (Molecular Devices). Data is analyzed for changes in maximal or total neurite length per neuron.

In vivo assays include animal models of various neurodegenerative diseases, such as spinal cord injury models, visual cortex plasticity models, and other models known in the art. Thus, regeneration and plasticity can be studied in models of plasticity following unilateral pyramidotomy and models or traumatic brain injury. Other models of neurodegeneration include mouse models of multiple sclerosis, such as experimental autoimmune encephalitis (EAE), models of amylotophic lateral sclerosis (ALS), such as the SODI mutant mouse, transgenic animal models of Alzheimer's disease, and animal models of Parkinson's disease.

### C. Making Antibodies Acting as Stimulators of Neuronal Regeneration

The antibodies identified by the binding and activity assays of the present invention can be produced by methods known in the art, including techniques of recombinant DNA technology.

i) Antigen Preparation

Soluble antigens or fragments thereof , optionally conjugated to other molecules, can be used as immunogens for generating antibodies. For transmembrane molecules, such as receptors, fragments of these (e.g. the extracellular domain of a receptor) can be used as the immunogen. Alternatively, cells expressing the transmembrane molecule can be used as the immunogen. Such cells can be derived from a natural source (e.g. cancer cell lines) or may be cells which have been transformed by recombinant techniques to express the transmembrane molecule. Other antigens and forms thereof useful for preparing antibodies will be apparent to those in the art.

(ii) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R₁N═C═NR, where R and R₁ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the scrum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(iii) Monoclonal Antibodies

Monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: to Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al, Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation to or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subloned by limiting dilution procedures and grown by standard methods (Goding, MonoclonalAntibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

In a further embodiment, antibodies or antibody fragments can be isolated from antibody phage libraries generated using the techniques described in McCafferty et al., Nature, 348:552-554 (1990).

Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iv) Humanized and Human Antibodies

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FIR) for the humanized antibody (Sims et al., J. Immunol., 151:2296 (1993); Chothia et al., J. Mol. Biol. 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immnol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J.sub.H) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al, Proc. Natl. Acad. Sci. USA, 90:2551 (1993); Jakobovits et al., Nature, 362:255-258 (1993): Bruggermann et al., Year in Immuno. 7:33 (1993); and Duchosal et al. Nature 355:258 (1992). Human antibodies can also be derived from phage-display libraries (Hoogenboom at al, J. Mol. Biol., 227:381 (1991); Marks et al, J. Mol. Biol., 222:581-597 (1991); Vaughan et al. Nature Biotech 14:309 (1996)). Generation of human antibodies from antibody phage display libraries is further described below.

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan at al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10: 163-167 (1992)). In another embodiment as described in the example below, the F(ab′)₂ is formed using the leucine zipper GCN4 to promote assembly of the F(ab′)₂ molecule. According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

(vi) Multispecific Antibodies

Multispecific antibodies have binding specificities for at least two different epitopes, where the epitopes are usually from different antigens. While such molecules normally will only bind two different epitopes (i.e. bispecific antibodies, BsAbs), antibodies with additional specificities such as trispecific antibodies are encompassed by this expression when used herein. Examples of BsAbs include those with one arm directed against PirB/LILRB and another arm directed against Nogo or MAG or OMgp. A further example of BsABs include those with one arm directed against PirB/LILRB and another arm directed against NgR.

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture or 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991). According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in WO96/27011, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′-SH fragments can also be directly recovered from E. coli, and can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al, J. Immunol, 152:5368 (1994).

Antibodies with more than two valencies are contemplated. For example, trispecific antibodies can be prepared. Tuft et al. J. Immunol. 147: 60 (1991).

(vii) Effector Function Engineering

It may be desirable to modify the antibody of the invention with respect to effector function, so as to enhance the effectiveness of the antibody. For example cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. Sec Stevenson et al Anti-Cancer Drug Design 3:219-230 (1989).

(viii) Antibody-Salvage Receptor Binding, Epitope Fusions.

In certain embodiments of the invention, it may be desirable to use an antibody fragment, rather than an intact antibody, to increase tumor penetration, for example. In this case, it may be desirable to modify the antibody fragment in order to increase its serum half life. This may be achieved, for example, by incorporation of a salvage receptor binding epitope into the antibody fragment (e.g. by mutation of the appropriate region in the antibody fragment or by incorporating the epitope into a peptide tag that is then fused to the antibody fragment at either end or in the middle, e.g., by DNA or peptide synthesis).

The salvage receptor binding epitope preferably constitutes a region wherein any one or more amino acid residues from one or two loops of a Fc domain are transferred to an analogous position of the antibody fragment. Even more preferably, three or more residues from one or two loops of the Fc domain are transferred. Still more preferred, the epitope is taken from the CH2 domain of the Fc region (e.g., of an IgG) and transferred to the CH1, CH3, or V.sub.H region, or more than one such region, of the antibody. Alternatively, the epitope is taken from the CH2 domain of the Fc region and transferred to the CL region or VL, region, or both, of the antibody fragment.

(ix) Other Covalent Modifications of Antibodies

Covalent modifications of antibodies are included within the scope of this invention. They may be made by chemical synthesis or by enzymatic or chemical cleavage of the antibody, if applicable. Other types of covalent modifications of the antibody are introduced into the molecule by reacting targeted amino acid residues of the antibody with an organic derivatizing agent that is capable of reacting with selected side chains or the N- or C-terminal residues. Examples of covalent modifications are described in U.S. Pat. No. 5,534,615, specifically incorporated herein by reference. A preferred type of covalent modification of the antibody comprises linking the antibody to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337.

(x) Generation of Antibodies from Synthetic Antibody Phage Libraries

In a preferred embodiment, the invention provides a method for generating and selecting novel antibodies using a unique phage display approach. The approach involves generation of synthetic antibody phage libraries based on single framework template, design of sufficient diversities within variable domains, display of polypeptides having the diversified variable domains, selection of candidate antibodies with high affinity to target the antigen, and isolation of the selected antibodies.

Details of the phage display methods can be found, for example, WO03/102157 published Dec. 11, 2003, the entire disclosure of which is expressly incorporated to herein by reference.

In one aspect, the antibody libraries used in the invention can be generated by mutating the solvent accessible and/or highly diverse positions in at least one CDR of an antibody variable domain. Some or all of the CDRs can be mutated using the methods provided herein. In some embodiments, it may be preferable to generate diverse antibody libraries by mutating positions in CDRH1, CDRH2 and CDRH3 to form a single library or by mutating positions in CDRL3 and CDRH3 to form a single library or by mutating positions in CDRL3 and CDRH1, CDRH2 and CDRH3 to form a single library.

A library of antibody variable domains can be generated, for example, having mutations in the solvent accessible and/or highly diverse positions of CDRH1, CDRH2 and CDRH3. Another library can be generated having mutations in CDRL1, CDRL2 and CDRL3. These libraries can also be used in conjunction with each other to generate binders of desired affinities. For example, after one or more rounds of selection of heavy chain libraries for binding to a target antigen, a light chain library can be replaced into the population of heavy chain binders for further rounds of selection to increase the affinity of the binders.

Preferably, a library is created by substitution of original amino acids with variant amino acids in the CDRH3 region of the variable region of the heavy chain sequence. The resulting library can contain a plurality of antibody sequences, wherein the sequence diversity is primarily in the CDRH3 region of the heavy chain sequence.

In one aspect, the library is created in the context of the humanized antibody 4D5 sequence, or the sequence of the framework amino acids of the humanized antibody 4D5 sequence. Preferably, the library is created by substitution of at least residues 95-100a of the heavy chain with amino acids encoded by the DVK codon set, wherein the DVK codon set is used to encode a set of variant amino acids for every one of these positions. An example of an oligonucleotide set that is useful for creating these substitutions comprises the sequence (DVK)₇. In some embodiments, a library is created by substitution of residues 95-100a with amino acids encoded by both DVK and NNK codon sets. An example of an oligonucleotide set that is useful for creating these substitutions comprises the sequence (DVK)₆(NNK). In another embodiment, a library is created by substitution of at least residues 95-100a with amino acids encoded by both DVK and NNK codon sets. An example of an oligonucleotide set that is useful for creating these substitutions comprises the sequence (DVK)₅ (NNK). Another example of an oligonucleotide set that is useful for creating these substitutions comprises the sequence (NNK)₆. Other examples of suitable oligonucleotide sequences can be determined by one skilled in the art according to the criteria described herein.

In another embodiment, different CDRH3 designs are utilized to isolate high affinity binders and to isolate binders for a variety of epitopes. The range of lengths of CDRH3 generated in this library is 11 to 13 amino acids, although lengths different from this can also be generated. H3 diversity can be expanded by using NNK, DVK and NVK codon sets, as well as more limited diversity at N and/or C-terminal.

Diversity can also be generated in CDRH1 and CDRH2. The designs of CDR-H1 and H2 diversities follow the strategy of targeting to mimic natural antibodies repertoire as described with modification that focus the diversity more closely matched to the natural diversity than previous design.

For diversity in CDRH3, multiple libraries can be constructed separately with different lengths of H3 and then combined to select for binders to target antigens. The multiple libraries can be pooled and sorted using solid support selection and solution sorting methods as described previously and herein below. Multiple sorting strategies may be employed. For example, one variation involves sorting on target bound to a solid, followed by sorting for a tag that may be present on the fusion polypeptide (eg. anti-gD tag) and followed by another sort on target bound to solid. Alternatively, the libraries can be sorted first on target bound to a solid surface, the eluted binders are then sorted using solution phase binding with decreasing concentrations of target antigen. Utilizing combinations of different sorting methods provides for minimization of selection of only highly expressed sequences and provides for selection of a number of different high affinity clones.

High affinity binders for the target antigen can be isolated from the libraries. Limiting diversity in the H1/H2 region decreases degeneracy about 10⁴ to 10⁵ fold and allowing more H3 diversity provides for more high affinity binders. Utilizing libraries with different types of diversity in CDRH3 (eg. utilizing DVK or NVT) provides for isolation of binders that may bind to different epitopes of a target antigen.

Of the binders isolated from the pooled libraries as described above, it has been discovered that affinity may be further improved by providing limited diversity in the light chain. Light chain diversity is generated in this embodiment as follows in CDRL1: amino acid position 28 is encoded by RDT: amino acid position 29 is encoded by RKT; amino acid position 30 is encoded by RVW: amino acid position 31 is encoded by ANW; amino acid position 32 is encoded by THT; optionally, amino acid position 33 is encoded by CTG; in CDRL2: amino acid position 50 is encoded by KBG; amino acid position 53 is encoded by AVC; and optionally, amino acid position 55 is encoded by GMA; in CDRL3: amino acid position 91 is encoded by TMT or SRT or both; amino acid position 92 is encoded by DMC; amino acid position 93 is encoded by RVT; amino acid position 94 is encoded by NHT; and amino acid position 96 is encoded by TWT or YKG or both.

In another embodiment, a library or libraries with diversity in CDRH1, CDRH2 and CDRH3 regions is generated. In this embodiment, diversity in CDRH3 is generated using a variety of lengths of H3 regions and using primarily codon sets XYZ and NNK or NAS. Libraries can be formed using individual oligonucleotides and pooled or oligonucleotides can be pooled to form a subset of libraries. The libraries of this embodiment can be sorted against target bound to solid. Clones isolated from multiple sorts can be screened for specificity and affinity using Et ISA assays. For specificity, the clones can be screened against the desired target antigens as well as other nontarget antigens. Those binders to the target antigen can then be screened for affinity in solution binding competition ELISA assay or spot competition assay. High affinity binders can be isolated from the library utilizing XYZ codon sets prepared as described above. These binders can be readily produced as antibodies or antigen binding fragments in high yield in cell culture.

In some embodiments, it may be desirable to generate libraries with a greater diversity in lengths of CDRH3 region. For example, it may be desirable to generate libraries with CDRH3 regions ranging from about 7 to 19 amino acids.

High affinity binders isolated from the libraries of these embodiments are readily produced in bacterial and eukaryotic cell culture in high yield. The vectors can be designed to readily remove sequences such as gD tags, viral coat protein component sequence, and/or to add in constant region sequences to provide for production of full length antibodies or antigen binding fragments in high yield.

A library with mutations in CDRH3 can be combined with a library containing variant versions of other CDRs, for example CDRL1, CDRL2, CDRL3, CDRH1 and/or CDRH2. Thus, for example, in one embodiment, a CDRH3 library is combined with a CDRL3 library created in the context of the humanized 4D5 antibody sequence with variant amino acids at positions 28, 29, 30, 31, and/or 32 using predetermined codon sets. In another embodiment, a library with mutations to the CDRH3 can be combined with a library comprising variant CDRH1 and/or CDRH2 heavy chain variable domains. In one embodiment, the CDRH1 library is created with the humanized antibody 4D5 sequence with variant amino acids at positions 28, 30, 31, 32 and 33. A CDRH2 library may be created with the sequence of humanized antibody 4D5 with variant amino acids at positions 50, 52, 53, 54, 56 and 58 using the predetermined codon sets.

(xi) Antibody Mutants

The novel antibodies generated from phage libraries can be further modified to generate antibody mutants with improved physical, chemical and or biological properties over the parent antibody. Where the assay used is a biological activity assay, the antibody mutant preferably has a biological activity in the assay of choice which is at least about 10 fold better, preferably at least about 20 fold better, more preferably at least about 50 fold better, and sometimes at least about 100 fold or 200 fold better, than the biological activity of the parent antibody in that assay. For example, an anti-PirB/LILRB antibody mutant preferably has a binding affinity for PirB/LILRB which is at least about 10 fold stronger, preferably at least about 20 fold stronger, more preferably at least about 50 fold stronger, and sometimes at least about 100 fold or 200 fold stronger, than the binding affinity of the parent antibody.

To generate the antibody mutant, one or more amino acid alterations (e.g. substitutions) are introduced in one or more of the hypervariable regions of the parent antibody. Alternatively, or in addition, one or more alterations (e.g. substitutions) of framework region residues may be introduced in the parent antibody where these result in an improvement in the binding affinity of the antibody mutant for the antigen from the second mammalian species. Examples of framework region residues to modify include those which non-covalently bind antigen directly (Amit et al. (1986) Science 233:747-753); interact with/effect the conformation of a CDR (Chothia et al. (1987) J. Mol. Biol. 196:901-917); and/or participate in the V_{L}-V_{H} interface (EP 239 400B1). In certain embodiments, modification of one or more of such framework region residues results in an enhancement of the binding affinity of the antibody for the antigen from the second mammalian species. For example, from about one to about five framework residues may be altered in this embodiment of the invention. Sometimes, this may be sufficient to yield an antibody mutant suitable for use in preclinical trials, even where none of the hype-variable region residues have been altered. Normally, however, the antibody mutant will comprise additional hypervariable region alteration(s).

The hypervariable region residues which are altered may be changed randomly, especially where the starting binding affinity of the parent antibody is such that such randomly produced antibody mutants can be readily screened.

One useful procedure for generating such antibody mutants is called “alanine scanning mutagenesis” (Cunningham and Wells (1989) Science 244:1081-1085). Here, one or more of the hypervariable region residue(s) are replaced by alanine or polyalanine residue(s) to affect the interaction of the amino acids with the antigen from the second mammalian species. Those hypervariable region residue(s) demonstrating functional sensitivity to the substitutions then are refined by introducing further or other mutations at or for the sites of substitution. Thus, while the site for introducing an amino acid sequence variation is predetermined, the nature of the mutation per se need not be predetermined. The ala-mutants produced this way are screened for their biological activity as described herein.

Normally one would start with a conservative substitution such as those shown below under the heading of “preferred substitutions”. If such substitutions result in a change in biological activity (e.g. binding affinity), then more substantial changes, denominated “exemplary substitutions” in the following table, or as further described below in reference to amino acid classes, are introduced and the products screened.

### Preferred Substitutions:

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; met; | ile |
| | ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

Even more substantial modifications in the antibodies biological properties are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr, asn, gin;

(3) acidic: asp, glu;

(4) basic: his, lys, arg;

(5) residues that influence chain orientation: gly, pro; and

(6) aromatic: trp, tyr, phe.

Non-conservative substitutions will entail exchanging a member of one of these classes for another class.

In another embodiment, the sites selected for modification are affinity matured using phage display (see above).

Nucleic acid molecules encoding amino acid sequence mutants are prepared by a variety of methods known in the art. These methods include, but are not limited to, oligonucleotide-mediated (or site-directed) mutagenesis, PCR mutagenesis, and cassette mutagenesis of an earlier prepared mutant or a non-mutant version of the parent antibody. The preferred method for making mutants is site directed mutagenesis (see, e.g., Kunkel (1985) Proc. Natl. Acad. Sci. USA 82:488).

In certain embodiments, the antibody mutant will only have a single hypervariable region residue substituted. In other embodiments, two or more of the hypervariable region residues of the parent antibody will have been substituted, e.g. from about two to about ten hypervariable region substitutions.

Ordinarily, the antibody mutant with improved biological properties will have an amino acid sequence having at least 75% amino acid sequence identity or similarity with the amino acid sequence of either the heavy or light chain variable domain of the parent antibody, more preferably at least 80%, more preferably at least 85%, more preferably at least 90%, and most preferably at least 95%. Identity or similarity with respect to this sequence is defined herein as the percentage of amino acid residues in the candidate sequence that are identical (i.e same residue) or similar (i.e. amino acid residue from the same group based on common side-chain properties, see above) with the parent-antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

Following production of the antibody mutant, the biological activity of that molecule relative to the parent antibody is determined. As noted above, this may involve determining the binding affinity and/or other biological activities of the antibody. In a preferred embodiment of the invention, a panel of antibody mutants is prepared and screened for binding affinity for the antigen or a fragment thereof. One or more of the antibody mutants selected from this initial screen are optionally subjected to one or more further biological activity assays to confirm that the antibody mutant(s) with enhanced binding affinity are indeed useful, e.g. for preclinical studies.

The antibody mutant(s) so selected may be subjected to further modifications, oftentimes depending on the intended use of the antibody. Such modifications may involve further alteration of the amino acid sequence, fusion to heterologous polypeptide(s) and/or covalent modifications such as those elaborated below. With respect to amino acid sequence alterations, exemplary modifications are elaborated above. For example, any cysteine residue not involved in maintaining the proper conformation of the antibody mutant also may be substituted, generally with serine, to improve the oxidative stability of the molecule and prevent aberrant cross linking. Conversely, cysteine bond(s) may be lidded to the antibody to improve its stability (particularly where the antibody is an antibody fragment such as an Fv fragment). Another type of amino acid mutant has an altered glycosylation pattern. This may be achieved by deleting one or more carbohydrate moieties found in the antibody, and/or adding one or more glycosylation sites that are not present in the antibody. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

(xii) Recombinant Production of Antibodies

For recombinant production of an antibody, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence (e.g. as described in U.S. Pat. No. 5,534,615, specifically incorporated herein by reference).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serrafia, e.g, Serratia marcescans, and Shigeila, as well as Bacilli such as B. subtilis and B. licheniformis (e.g., B. licheniformis 41P disclosed in DD 266,710 published Apr. 12, 1989), Pseudomonas such as P. aeruginosa, and Stieptomyces. One preferred E. coli cloning host is E. coli 294 (ATCC 31,446), although other strains such as E. coli B, E. coli X 1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATrCC 16.045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available e.g., the L-1 variant of Autographa calilornica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651): human embryonic kidney line (293 or 293 cells subloned for growth in suspension culture, Graham et al, J. Gen Virol. 36:59 (1977)); baby hamster kidney cells (BHK, ATCC CCL 10); Chinese hamster ovary cells/-DHFR(CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC. CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce the antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed cells, is removed, (or example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human gamma. 1, .gamma.2, or .gamma.4 heavy chains (Lindmark et al., J. Immunol. Meth. 62:1-13 (1983)). Protein G is recommended for all mouse isotypes and for human .gamma.3 (Guss et al., EMBO J. 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH 3 domain, the Bakerbond ABX™ M resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipiation are also available depending on the antibody to be recovered.

### D. Uses of Stimulators of Neuronal Regeneration

The molecules identified in the screening assays of the present invention are believed to find use as agents for enhancing the survival or inducing the outgrowth of nerve cells. They are, therefore, useful in the therapy of degenerative disorders of the nervous system (“neurodegenerative diseases”), including, for example, peripheral nerve damage caused by physical injury (e.g., burns, wounds) or disease states such as diabetes, kidney dysfunction or by the toxic effects of chemotherapeutics used to treat cancer and AIDS; physical damage to the central nervous system (spinal cord and brain); brain damage associated with stroke; and neurological disorders relating to neurodegeneration, such as, for example, trigeminal neuralgia, glossopharyngeal neuralgia, Bell's Palsy, myasthenia gravis, muscular dystrophy, amyotrophic lateral sclerosis (ALS), progressive muscular atrophy, progressive bulbar inherited muscular atrophy, herniated, ruptured or prolapsed invertebrate disk syndromes, cervical spondylosis, plexus disorders, thoracic outlet destruction syndromes, peripheral neuropathies such as those caused by lead, dapsone, ticks, prophyria, Gullain-Barre syndrome, Alzheimer's disease, Huntington's Disease, or Parkinson's disease.

The compounds identified herein are also useful as components of culture media for use in culturing nerve cells it vitro.

Finally, preparations comprising compounds identified by the assays herein are useful as standards in competitive binding assays when labeled with radioiodine, enzymes, fluorophores, spin labels, and the like.

Therapeutic formulations of the compounds herein are prepared for storage by mixing the compound identified (such as an antibody) having the desired degree of purity with optional physiologically acceptable carriers, excipients or stabilizers (Remington's Pharmaceutical Sciences, supra), in the form of lyophilized cake or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or PEG.

Compounds to be used for in vivo administration must be sterile. This is readily accomplished by filtration through sterile filtration membranes, prior to or following lyophilization and reconstitution.

Therapeutic compositions may be placed into a container having a sterile access poll, for example, an intravenous solution bag or vial having a stopper pierceable by a hypodermic injection needle.

The compounds identified by the assays of the present invention may be optionally combined with or administered in concert with neurotrophic factors including NGF, NT-3, and/or BDNF and used with other conventional therapies for degenerative nervous disorders.

The route of administration is in accord with known methods, e.g. injection or infusion by intravenous, intraperitoneal, intracerebral, intramuscular, intraocular, intraarterial or intralesional routes, topical administration, or by sustained release systems as noted below.

For intracerebral use, the compounds may be administered continuously by infusion into the fluid reservoirs of the CNS, although bolus injection is acceptable. The compounds are preferably administered into the ventricles of the brain or otherwise introduced into the CNS or spinal fluid. Administration may be performed by an indwelling catheter using a continuous administration means such as a pump, or it can be administered by implantation, e.g., intracerebral implantation, of a sustained-release vehicle. More specifically, the compounds can be injected through chronically implanted cannulas or chronically infused with the help of osmotic minipumps. Subcutaneous pumps are available that deliver proteins through a small tubing to the cerebral ventricles. Highly sophisticated pumps can be refilled through the skin and their delivery rate can be set without surgical intervention. Examples of suitable administration protocols and delivery systems involving a subcutaneous pump device or continuous intracerebroventricular infusion through a totally implanted drug delivery system are those used for the administration of dopamine, dopamine agonists, and cholinergic agonists to Alzheimer patients and animal models for Parkinson's disease described by Harbaugh, J. Neural Transm. Suppl., 24:271 (1987); and DeYebenes, et al., Mov. Disord. 2:143 (1987).

Suitable examples of sustained release preparations include semipermeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman, et al., 1983, Biopolymers 22:547), poly(2-hydroxyethyl-methacrylate) (Langer, et al., 1981, J. Biomed. Mater. Res. 15:167; Langer, 1982, Chem. Tech. 12:98), ethylene vinyl acetate (Langer, et al., Id.) or poly-D-(−)-3-hydroxybutyric acid (EP 133,988A). Sustained release compositions also include liposomally entrapped compounds, which can be prepared by methods known per se. (Epstein, et al., Proc. Natl. Acad. Sci. 82:3688 (1985); Hwang, et al., Proc. Natl. Acad. Sci. USA 77:4030 (1980); U.S. Pat. Nos. 4,485,045 and 4,544,545; and EP 102,324A). Ordinarily the liposomes are of the small (about 200-800 Angstroms) unilamelar type in which the lipid content is greater than about 30 mol·% cholesterol, the selected proportion being adjusted for the optimal therapy.

An effective amount of an active compound to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. A typical daily dosage might range from about 1 μg/kg to up to 100 mg/kg or more, depending on the factors mentioned above. Typically, the clinician will administer an active compound until a dosage is reached that repairs, maintains, and, optimally, reestablishes neuron function. The progress of this therapy is easily monitored by conventional assays.

Further details of the invention are illustrated by the following non-limiting examples.

### Example 1

### Expression Cloning LILRB2

To identify novel receptors for inhibitory myelin proteins, an expression cloning approach was taken. As bait, constructs were generated that fused Alkaline Phosphatase (AP) to the N- and/or C-terminus of the following characterized myelin inhibitors (human cDNA used): Nogo66, two additional inhibitory domains of NogoA (NiR<delta>D2 and NiG<delta>20) (Oertle T, J. Neurosci. 2003, 23(13): 5393-406), MAG, and OMgp. These constructs were transfected into 293 cells to produce conditioned medium (in DMEM/2% FBS) containing the bait proteins. The cDNA library used in the screen was comprised of full-length human cDNA clones in expression-ready vectors generated by Origene. These cDNAs were compiled, arrayed, and pooled. Pools of approximately 100 cDNA's were transiently transfected into COS7 cells.

In particular, on Day 1, COS7 cells were plated at a density of 85,000 cells per well in 12-well plates. On Day 2, 1 mg of pooled cDNA's were transfected per well using the lipid-based transfection reagent FuGENE 6 (Roche). On Day 4, screening was performed. Briefly, culture medium was removed from cells and replaced with 0.5 ml of 293 cell-conditioned medium containing AP-fusion bait proteins (20-50 nM). Cells were incubated at room temperature for 90 minutes. The cells were then washed 3 times with phosphate-buffered saline (PBS), fixed for 7 minutes with 4% paraformaldehyde, washed 3 times in HEPES-buffered saline (HBS), and heat inactivated at 65° C. for 90 minutes to destroy endogenous AP activity. The cells were washed once in AP Buffer (100 mM NaCl, 5 mM MgCl₂, 100 mM Tris pH 9.5), and incubated in chromogenic substrate (Western Blue, Promega), and analyzed for presence of reaction product one hour after incubation, and again after overnight incubation. Positive cells were identified by the presence of dark blue precipitate over the surface of the membrane. Positive pools were further broken down to identify individual positive clones by subsequent rounds of screening.

From the screening, the following positive hits were identified:

MAG-AP bait yielded 4 positive hits. One was the previously characterized Nogo Receptor (Fournier et al., Nature 409, 342-346 (2001). Two of these hits were glycolytic processing enzymes, and deemed unlikely to be of relevance. The fourth was annotated as “Homo sapiens hypothetical protein from clone 643 (LOC57228), mRNA”. Closer analysis of the cDNA revealed an alternative ORF that was homologous to the previously described protein SMAG.

AP-Nogo66 bait yielded 2 positive hits. One was the previously characterized Nogo Receptor. The other was “Homo sapiens leukocyte immunoglobulin-like receptor, subfamily B (with TM and ITIM domains), member 2 (LILRB2), mRNA” (SEQ ID NO: 2). This gene is also known by multiple alternative nomenclatures, including MIG10, ILT4, and LIR2 (.Kubagawa et al., Proc. Natl. Acad. Sci. USA 94:5261-6 (1997); Colonna et al., J. Exp. Med. 186:1809-18 (1997)).

### Example 2

### AP-Nogo66 Binds PirB and LILRB2

To confirm Nogo66 binding to LILRB2 (SEQ ID NO: 2), and to test if Nogo66 binds to the murine orthologue PirB (SEQ ID NO: 1), binding assays similar to those described in Example 1 were carried out. Briefly, COS7 cells were transfected with cDNA's encoding PirB, LILRB2, or NgR as a positive control. 48 hours following transfection, cells were incubated with 293 cell-conditioned medium containing AP-Nogo66 for 90 minutes at RT. Cells were washed extensively, fixed, and endogenous AP activity was neutralized by heat inactivation. Cells were then reacted with chromogenic substrate (Western Blue, Promega).

As shown in FIG. 1, a strong positive signal was detected on both PirB- and LILRB2-expressing cells.

### Example 3

### MAG Binds PirB and LILRB2

To test whether or not MAG also binds to PirB and LILRB2, binding assays were performed with MAG-Fc, which is believed to be more bioactive than MAG-AP. COS7 cells were transfected with cDNA's encoding PirB, LILRB2, or mSMAGP as a positive control. 48 hours following transfection, cells were incubated with 293 cell-conditioned medium containing MAG-Fe for 90 minutes at RT. Cells were washed four times with Hank's Buffered Saline Solution (HBSS), fixed for five minutes with 2% paraformaldehyde, washed four times with HBSS, and blocked for 15 minutes with 10% heat-inactivated goat serum (HIGS) in HBSS. Cells were then incubated for one hour with anti-human Fe antibody (1:500, Jackson Immunochemicals), washed with 10% HIGS in PBS, and incubated with secondary antibody (AlexaFluor 568-conjugated goat anti-mouse, Molecular Probes). Cells were washed with PBS, coverslipped, and immuno-fluorescence was detected using an inverted fluorescence microscope.

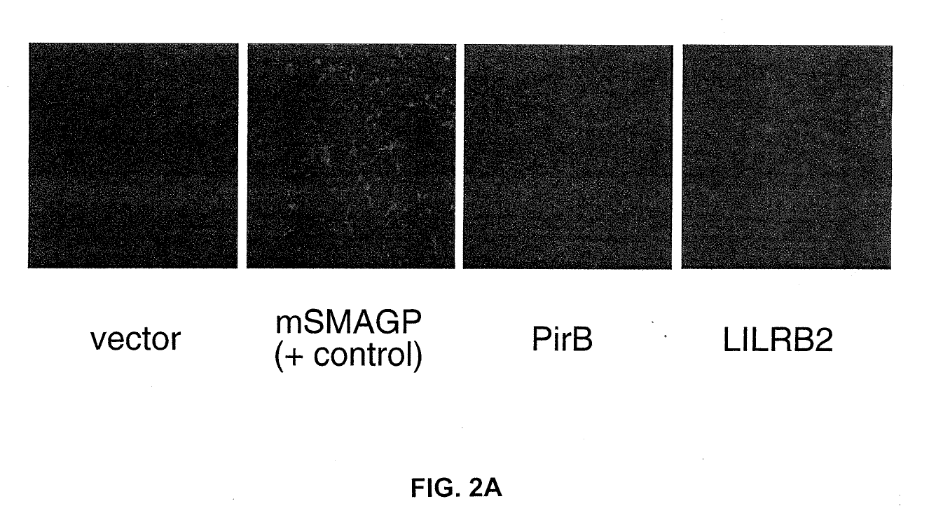

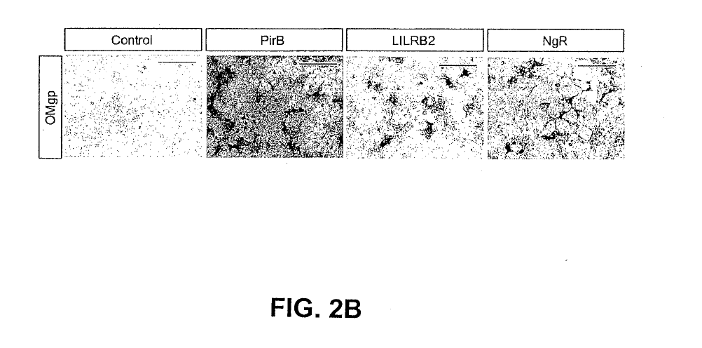

The results are shown in FIG. 2A. Compared to control cells, both PirB- and LILRB2-expressing cells show binding to MAG-FC. In this example, mSMAGP-expressing cells served as a positive control for binding.

### Example 4

### AP-OMgp Binds PirB and LILRB2

To test whether or not AP-OMgp binds PirB and LILRB2, binding assays were performed with AP-OMgp, essentially as described in Examples 2 and 3. COS7 cells were transfected with empty vector, or PirB, LIRB2, or BGR cDNA. After 48 hours, cells were incubated with AP-OMgp, washed extensively, and bound ligand was detected directly by reaction with a chromogenic substrate (Western Blue, Promega).

The results are shown in FIG. 2B. Compared to control cells, both PirB- and LILRB2-expressing cells show binding to OMgp.

### Example 5

### PirB is Expressed in the Nervous System

To address whether or not PirB is expressed in the nervous system, RT-PCR analysis was performed on mRNA isolated from different neural tissues. P7 cerebellum, P10 Dorsal Root Ganglia (DRG), adult brain, and adult spleen (positive control) were dissected from CD1 mice and immediately placed in RNAlater (Ambion). mRNA was extracted using the RNEasy isolation kit (Qiagen). cDNA was generated from mRNA using Invitrogen's Superscript III First Strand Synthesis system. RT-PCR was then performed using primers specific for PirB 5′TGAAGGCTCTCATTGGAGTGTCTG3′ (SEQ ID NO: 20) and 5′GGCATAGGTCACATCCTGGGAC3′ (SEQ NO: 3), primers cross-reactive with different members of the PirA subfamily (5′GTCTCAGAAACCATTGAATCC3′ (SEQ ID NO: 4) and 5′GACAGAAAACTTTGGGTCATCAG3′ (SEQ ID NO: 5)), or primers specific for mouse SMAGP (5′CCCTCAGCAACGATGAACAACC3′ (SEQ ID NO: 6) and 5′TGGACCCTGGAGTCAGTGATTC3′(SEQ ID NO: 7)).

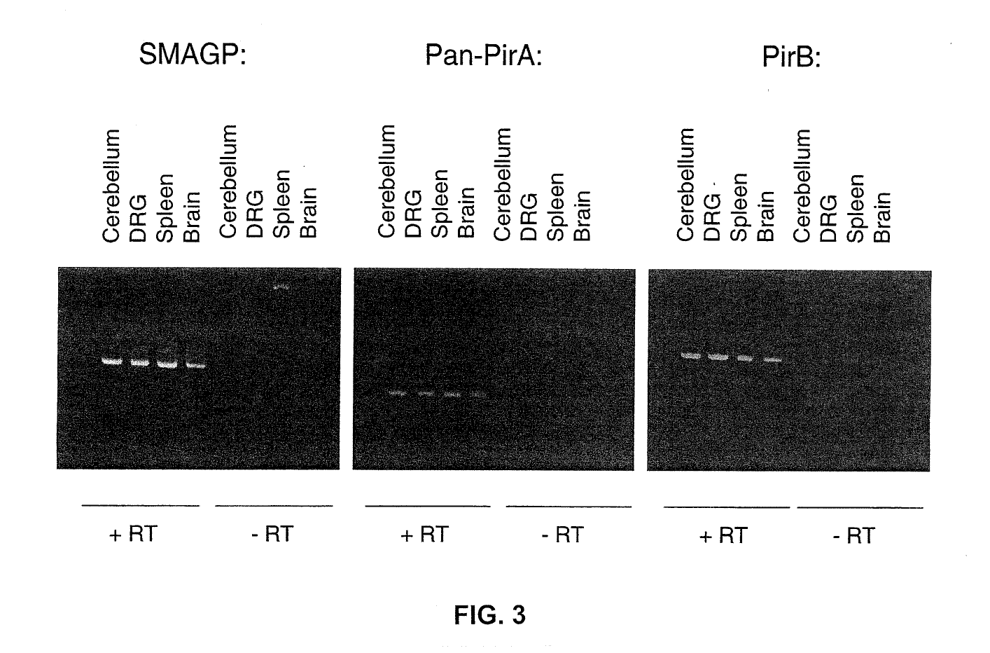

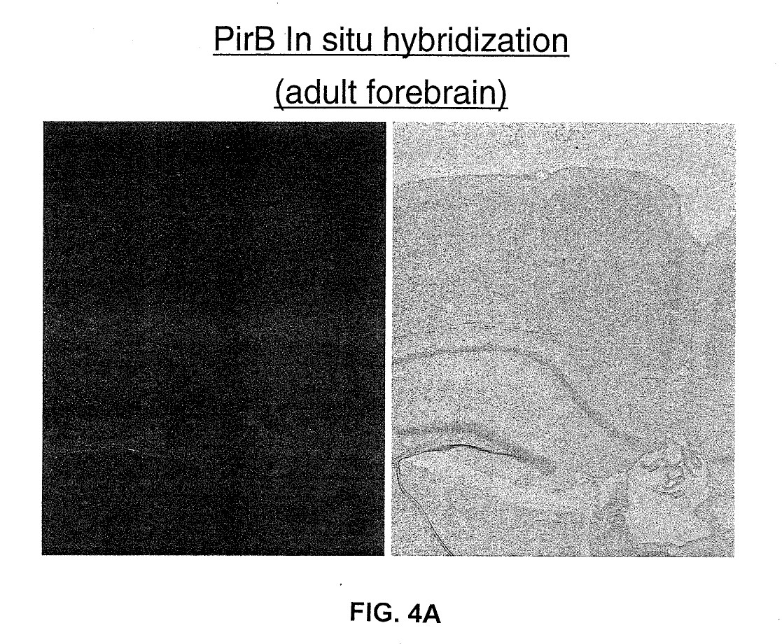

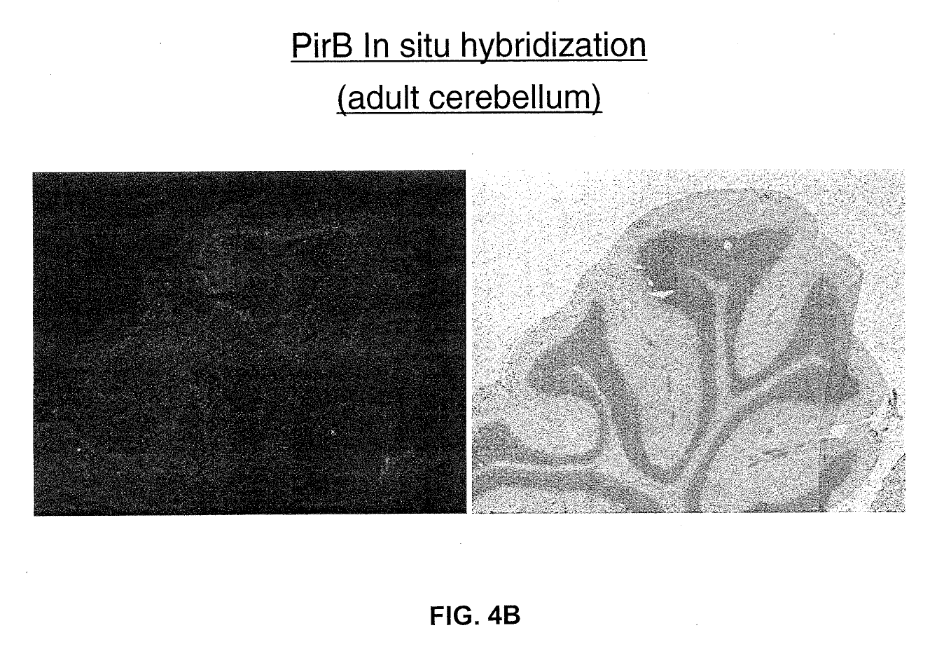

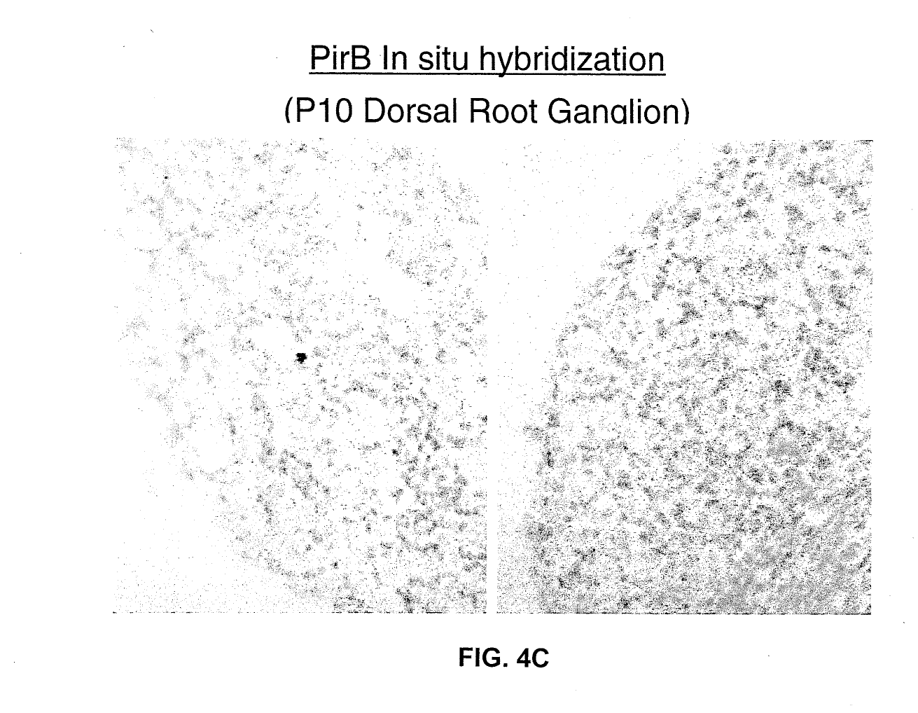

As shown in FIG. 3, RT-PCR analysis revealed that both PirB and PirA isoforms could be detected in cerebellum, DRG, and brain.

### Example 6

### In Situ Hybridization Results

To analyze the neuronal expression of PirB in greater detail, in situ hybridization analyses were performed. Adult or postnatal mice were perfused, and then brain and DRG were dissected out and post-fixed overnight. Tissues were subsequently cryo-protected in 30% sucrose, and embedded and frozen in O.C.T. Compound (Tissue-Tek). Frozen tissues were sectioned at 12 micron thickness. Radioactive probes were prepared using the MAXIscript in vitro transcription kit (Ambion), following manufacturer's protocols. In situ hybridization was carried out using the mRNAlocator ISH kit (Ambion), according to the manufacturer's protocols.

The PirB probe was designed to hybridize to nucleotides #1922-2385, which are in the transmembrane and intracellular domain unique to PirB. Primers used to amplify this region were 5′TGAAGGCTCTCATTGGAGTGTCTG3′ (SEQ ID NO: 8) and 5′GGCATAGGTCACATCCTGGGAC3′ (SEQ ID NO: 9). The 464 bp fragment was cloned into pCRII-TOPO (Invitrogen).

The results of in situ hybridization are shown in 1FIGS. 4A-4C. Positive hybridization signal is seen throughout the cortex, in the hippocampus, in the cerebellum, and in cells of the DRG.

### Example 7

### Nogo66 Inhibition of Axonal Growth and its Rescue by PirB ECD in Cerebellar Granule Neurons

In this experiment, Nogo-66's ability to inhibit axonal growth was confirmed. Furthermore, PirB extracellular domain constructs were tested for their ability to interfere Nogo-66 inhibitory activity.

AP-Nogo66 was generated by cloning the 66 amino acid inhibitory loop of NogoA downstream of the human placental alkaline phosphatase (AP) gene. The construct was FLAG-tagged at the N-terminus to allow purification, and inserted into a pRK vector backbone (Genentech). To generate PirB extracellular domain (ECD) proteins, amino acids #1-638 of PirB were cloned into pRK expression vectors upstream of either an 8-His tag (PirBHis) or human Ic (PirBFc). These expression constructs were transiently transfected into CHO cells, and the secreted proteins were purified from the conditioned medium by affinity chromatography.

Cerebellar granule neurons (CGN) were isolated from P7 CD1 mice, and cultured on immobilized AP-Nogo66 protein for inhibition assays. Briefly, 96 well plates pre-coated with poly-D-lysine (Biocoat, Becton Dickinson) were spotted with recombinant AP-Nogo66 (180 or 300 ng/3ul spot). The AP-Nogo66 was coated alone, or mixed with an excess of either PirBFc (850 ng/3 ul spot) or PirBHis (1000 ng/3 ul spot). This resulted in spots containing a 2-4 fold molar excess of PirB ECD. Spotted proteins were allowed to adhere for 2 hours, and then plates were treated with 10 ug/ml laminin (Invitrogen) for 2 hr. Mouse P7 cerebellar cells were prepared as described (Zheng et al., 2005) and plated at a density of 2×10⁴ cells/well. Cultures were incubated at 37° for 22 hrs, fixed with 4% paraformaldehyde/4% sucrose, and stained with an anti-tubulin antibody (TuJ1, Covance). Images were captured with the ImageXpress imaging system (Molecular Devices).

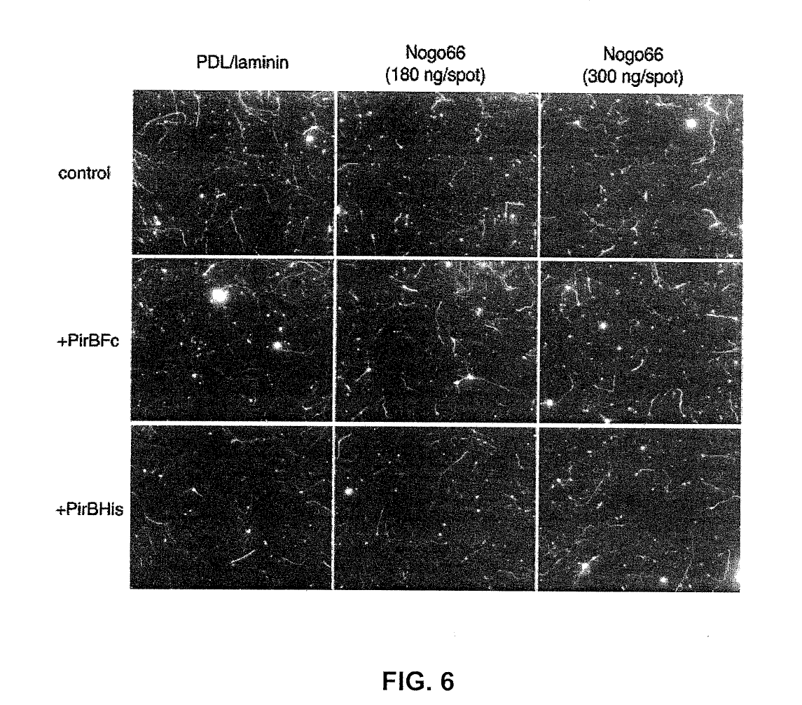

As shown in FIG. 6, AP-Nogo66 strongly inhibits axon outgrowth from P7 cerebellar neurons. The presence of an excess of PirB ECD, either with PirBFc or PirBHis, significantly reduced this inhibition. Inclusion of other control proteins (Fc or Robo4Fc) with AP-Nogo66 did not reduce inhibition.

### Example 8

### Co-Immunoprecipitation of PirB and NgR

This experiment explore the relationship and potential interaction of PirB and NgR when co-expressed in host cells in vitro.

COS7 cells were transiently transfected with a control vector, full-length PirB, or a mixture of full-length PirB and full-length NgR. 48 hours after transfection, cells were lysed with Cell Lysis Buffer (Cell Signaling Technology) and lysates were immunoprecipitated with anti-PirA/B (6C1, Pharmingen). Samples were separated by SDS-PAGE, transferred to nitrocellulose, and probed with anti-NgR (Alpha Diagnostics International).

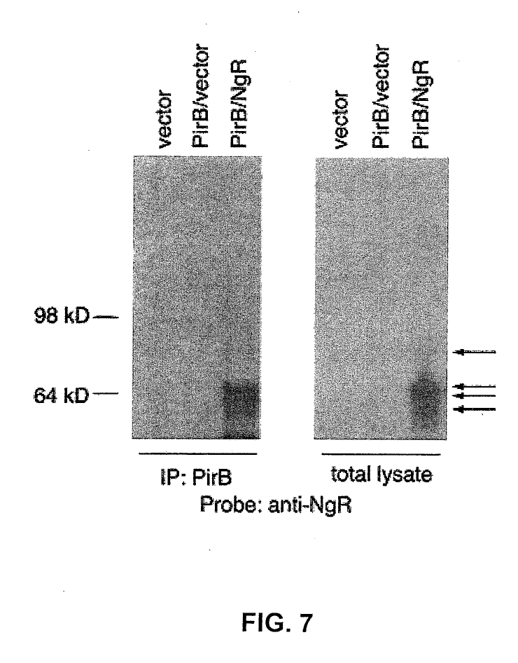

As shown in FIG. 7, NgR was robustly co-precipitated with PirB (left panel). The right panel shows total protein from whole cell lysates immunoblotted with anti-NgR. The multiple bands (arrows) represent NgR processed by glycosylation to varying extents.

### Example 9

### PirB Antibodies and their Use to Interfere with Nogo66 Axonal Growth Inhibitory Activity

In this experiment, antibodies against PirB were tested for their ability to interfere with Nogo-66 induced inhibition of axonal growth.

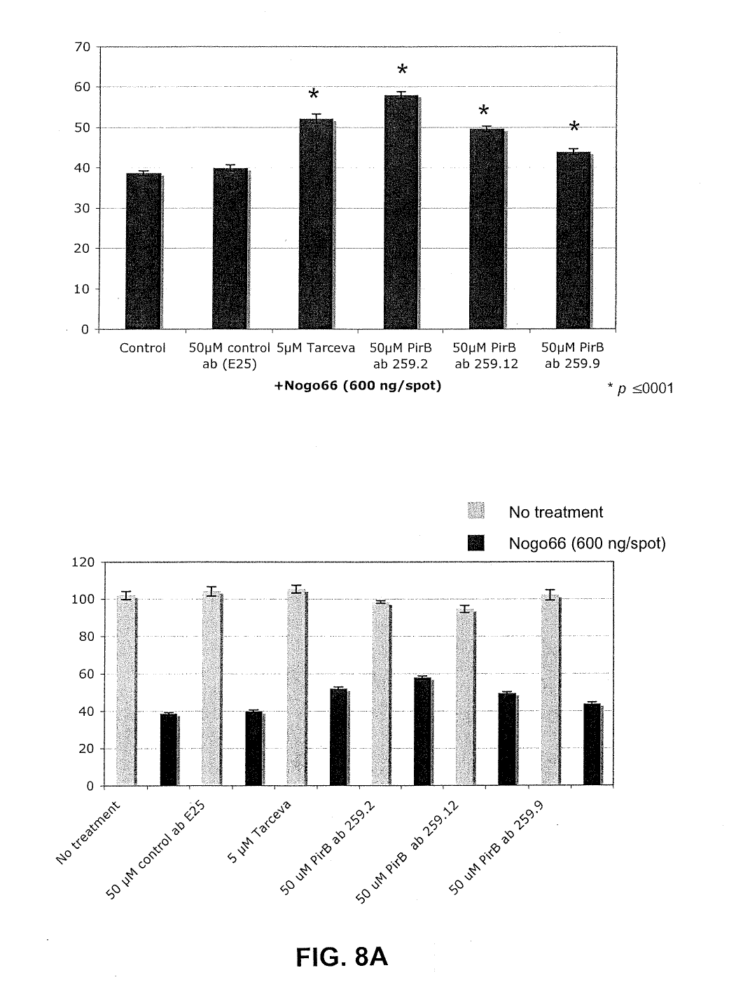

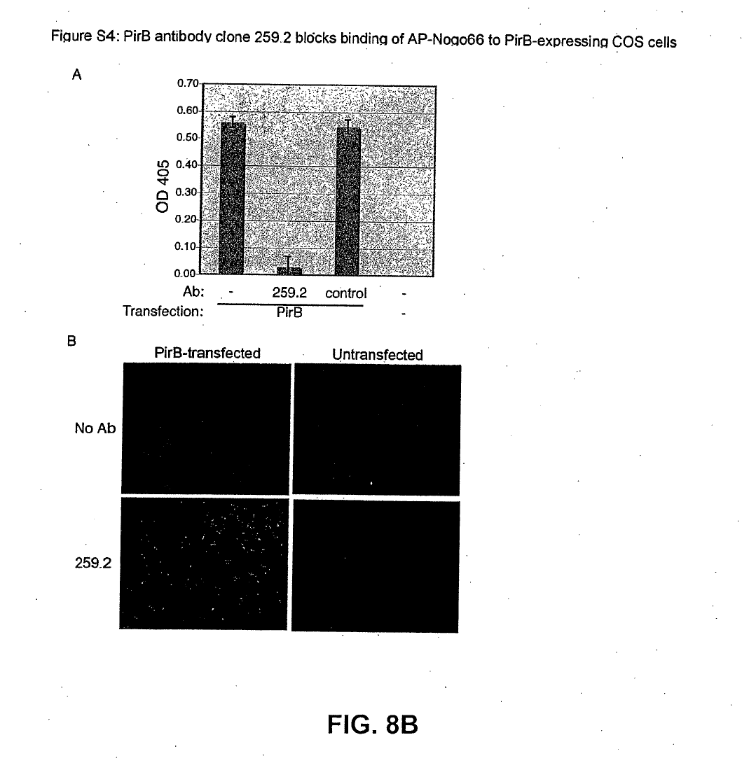

Cerebellar granule neurons (CGN) were isolated from P7 CD1 mice and cultured on immobilized AP-Nogo66 protein for inhibition assays, as described in the previous examples. Function-blocking antibodies were generated by screening PirB ECD against a human synthetic antibody phage library, essentially as described in Liang et al., J. Mol. Biol. 366(3):815-819 (2006), which is incorporated by reference herein in its entirety. Clones were selected for their ability to compete with AP-Nogo66 binding to PirB. In this experiment, anti-PirB or control antibodies were incubated with neurons grown on AP-Nogo66. As shown in FIG. 8A, the anti-PirB antibodies led to a significant decrease in inhibition by AP-Nogo66.

### Example 10

### PirB Antibody Blocks Binding of AO-Nogo66 to PirB Expressing COS Cells

Quantitative binding of AP-Nogo66 to COS7 cells transfected with pirB was carried out in the presence of different antibodies. As shown in FIG. 8B, panel (A), anti-PirB. 1 antibody clone 259.2, but not control antibody (E25), blocks binding of AP-Nogo66 (50 μM) to PirB. Panel (B) shows that clone 259.2 binds specifically to PirB-transfected COS cells.

All references cited throughout the disclosure are hereby expressly incorporated by reference in their entirety

While the present invention has been described with reference to what are considered to be the specific embodiments, it is to be understood that the invention is not limited to such embodiments. To the contrary, the invention is intended to cover various modifications and equivalents included within the spirit and scope of the appended claims.

1. A method for identifying a PirB/LILRB antagonist comprising contacting a candidate agent with a complex comprising PirB/LILRB and myelin or a myelin-associated protein, or a fragment thereof, and detecting the ability of said candidate agent to inhibit the interaction between PirB/LILRB and said myelin or myelin-associated protein, or fragment thereof, wherein the candidate agent is identified as an antagonist if the interaction is inhibited.

2. The method of claim 1 wherein the interaction is binding.

3. The method of claim 1 wherein the interaction is cellular signaling.

4. The method of claim 3 wherein said cellular signaling results in the inhibition of axonal outgrowth or neuronal regeneration.

5. The method of claim 1 wherein the myelin-associated protein is selected from the group consisting of Nogo, MAG and OMgp.

6. The method of claim 5 wherein said PirB/LILRB is selected from the group consisting of LILRB 1, ILRB2, LILRB3, and LILRB5.

7. The method of claim 6 wherein said PirB/LILRB is selected from the group consisting of LILRB2, transcript variant 1 (SEQ ID NO: 2), LILRB2, transcript variant 2 (SEQ ID NO: 14), LILRB1, transcript variant 1 (SEQ ID NO: 10), LILRB1, transcript variant 2 (SEQ ID NO: 11), LILRB1, transcript variant 3 (SEQ ID NO: 12), LILRB1, transcript variant 4 (SEQ ID NO: 13), LILRB3, transcript variant 1 (SEQ ID NO: 15), LILRB3, transcript variant 2 (SEQ ID NO: 16), LILRB5, transcript variant 1 (SEQ ID NO: 17). LILRB5, transcript variant 2 (SEQ ID NO: 18), and LILRB5, transcript variant 3 (SEQ ID NO: 19).

8. The method of claim 7 wherein said PirB is LILRB2, transcript variant 1 (SEQ ID NO: 2) or LILRB2, transcript variant 2 (SEQ ID NO: 14).

9. The method of claim 5 wherein the complex further comprises NgR.

10. The method of claim 1 wherein the candidate agent is selected from the group consisting of antibodies, polypeptides, peptides, nucleic acids, short interfering RNAs (siRNAs), small organic molecules, polysaccharides and polynucleotides.

11. The method of claim 10 wherein the candidate agent is an antibody.

12. The method of claim 11 wherein said antibody specifically binds PirB/LILRB.

13. The method of claim 12 wherein said antibody specifically binds an LILRB2.

14. The method of claim 12 wherein said antibody is a monoclonal antibody.

15. The method of claim 12 wherein said antibody in a chimeric antibody.

16. The method of claim 12 wherein said antibody is a humanized antibody.

17. The method of claim 12 wherein said antibody is a human antibody.

18. The method of claim 12 wherein said antibody is an antigen-binding fragment.

19. The method of claim 18 wherein said antibody fragment is selected from the group consisting of Fv, Fab, Fab′, and F(ab′)₂ fragments.

20. The method of claim 10 wherein the candidate agent is a short-interfering RNA (siRNA).

21. The method of claim 1 wherein at least one of said PirB/LILRB and said myelin or myelin-associated protein, or fragment thereof, is immobilized.

22. The method of claim 1 which is a cell-based assay.

23. The method of claim 22 wherein said cell-based assay comprises culturing neuronal cells with said myelin or myelin-associated protein, or fragment thereof in the presence and absence of said candidate agent and determining the change in neurite length, wherein said candidate agent is identified as an antagonist when the neurite length is longer in the presence of said candidate agent.

24. The method of claim 23 wherein said neuronal cells are primary neurons.

25. The method of claim 23 wherein said neuronal cells are derived from embryonic stem (ES) cells or cell lines.

26. The method of claim 25 wherein said neuronal cells are derived from neuroblastoma.

27. The method of claim 23 wherein said neuronal cells are selected from the group consisting of cerebellar granule neurons, dorsal root ganglion neurons, and cortical neurons.

28. The method of any one of claims 1 to 27 further comprising the step of using the antagonist identified to enhance neurite outgrowth, and/or promote neuronal growth, repair and/or regeneration.

29. The method of any one of claims 1 to 27 further comprising the step of administering the antagonist identified to a subject with a disease or condition benefiting from the enhancement of neurite outgrowth promotion of neuronal growth, repair or regeneration.

30. The method of claim 29 wherein said disease or condition is a neurological disorders.

31. The method of claim 30 wherein said neurological disorder is characterized by a physically damaged nerve.

32. The method of claim 30 wherein said neurological disorder is selected from the group consisting of peripheral nerve damage caused by physical injury, diabetes; physical damage to the central nervous system; brain damage associated with stroke, trigeminal neuralgia, glossopharyngeal neuralgia, Bell's Palsy, myasthenia gravis, muscular dystrophy, amyotrophic lateral sclerosis (ALS), progressive muscular atrophy, progressive bulbar inherited muscular atrophy, herniated, ruptured and prolapsed invertebrate disk syndromes, cervical spondylosis, plexus disorders, thoracic outlet destruction syndromes, peripheral neuropathies, prophyria, Gullain-Barre syndrome, Alzheimer's disease, Huntington's Disease, and Parkinson's disease.

33. An agent identified by any one of the methods of claims 1 to 30.

34. The agent of claim 33 selected from the group consisting of antibodies, polypeptides, peptides, nucleic acids, small organic molecules, polysaccharides and polynucleotides.

35. The agent of claim 34 which is an antibody.

36. The agent of claim 34 which is a short-interfering RNA (siRNA).

37. A composition comprising an agent of claim 33 for stimulation of neuronal regeneration.

38. A kit comprising an agent of claim 33 and instructions for neuronal regeneration.

39. A method of reducing the inhibition of axonal growth in a neuron of the CNS, comprising contacting said neuron with a PirB/LILRB antagonist identified according to claims 1 to 30.

40. A method for promoting axonal growth in a neuron of the CNS, comprising contacting said neuron with a PirB/LILRB antagonist identified according to claims 1 to 30.

41. A method for treating neural injury in a subject, comprising administering to said subject a PirB/LILRB antagonist identified according to claims 1 to 30.

42. A method for maintaining the viability of a neuron in the CNS, comprising contacting said neuron with a PirB/LILRB antagonist identified according to claims 1 to 30.

43. Use of a complex of PirB/LILRB and myelin or a myelin-associated protein, or a fragment thereof to identify a PirB/LILRB antagonist.

44. Use of a PirB/LILRB antagonist in the preparation of a medicament for the treatment of a disease or condition benefiting from the enhancement of neurite outgrowth, promotion of neuronal growth, repair or regeneration.

45. Use of a PirB/LILRB antagonist in the preparation of a medicament for the treatment of a neurological disorder.

46. The use according to claim 45 wherein said neurological disorder is characterized by a physically damaged nerve.

47. The use according to claim 45 wherein said neurological disorder is selected from the group consisting of peripheral nerve damage caused by physical injury, diabetes; physical damage to the central nervous system; brain damage associated with stroke, trigeminal neuralgia, glossopharyngeal neuralgia, Bell's Palsy, myasthenia gravis, muscular dystrophy, amyotrophic lateral sclerosis (ALS), progressive muscular atrophy, progressive bulbar inherited muscular atrophy, herniated, ruptured and prolapsed invertebrate disk syndromes, cervical spondylosis, plexus disorders, thoracic outlet destruction syndromes, peripheral neuropathies, prophyria, Gullain-Barre syndrome, Alzheimer's disease, Huntington's Disease, and Parkinson's disease.

48. A PirB/LILRB antagonist for use in the treatment of a disease or condition benefiting from the enhancement of neurite outgrowth, promotion of neuronal growth, repair or regeneration.

49. A PirB/LILRB antagonist for use in the treatment of a neurological disorder.

50. The PirB/LILRB antagonist of claim 50 wherein said neurological disorder is characterized by a physically damaged nerve.

51. The PirB/LILRB antagonist of claim 50 wherein said neurological disorder is selected from the group consisting of peripheral nerve damage caused by physical injury, diabetes; physical damage to the central nervous system; brain damage associated with stroke, trigeminal neuralgia, glossopharyngeal neuralgia, Bell's Palsy, myasthenia gravis, muscular dystrophy, amyotrophic lateral sclerosis (ALS), progressive muscular atrophy, progressive bulbar inherited muscular atrophy, herniated, ruptured and prolapsed invertebrate disk syndromes, cervical spondylosis, plexus disorders, thoracic outlet destruction syndromes, peripheral neuropathies, prophyria, Gullain-Barre syndrome, Alzheimer's disease, Huntington's Disease, and Parkinson's disease.
